(19) United States
(12) Patent Application Publication
(10) Pub. No.: US 2014/0308520 A1
(43) Pub. Date: Oct. 16, 2014

(54) Methods for the Bio-programmable Crystallization of Multi-component Functional Nanoparticle Systems

(71) Applicants: Yugang Zhang, Middle Island, NY (US); Fang Lu, Middle Island, NY (US); Oleg Gang, Setauket, NY (US); Daniel Van Der Lelie, Chapel Hill, NC (US)

(72) Inventors: Yugang Zhang, Middle Island, NY (US); Fang Lu, Middle Island, NY (US); Oleg Gang, Setauket, NY (US); Daniel Van Der Lelie, Chapel Hill, NC (US)

(73) Assignee: BROOKHAVEN SCIENCE ASSOCIATES, LLC, Upton, NY (US)

(21) Appl. No.: 14/111,732

(22) PCT Filed: Apr. 12, 2012
(86) PCT No.: PCT/US12/33380

§ 371 Date: Feb. 25, 2014

Related U.S. Application Data
(60) Provisional application No. 61/475,172, filed on Apr. 13, 2011.

(51) Int. Cl. C07K 14/36; C07K 19/00

(52) U.S. Cl. CPC C07K 14/36; C07K 19/00
USPC 428/402; 530/358

(57) ABSTRACT

The bio-programmable crystallization of multi-component functional nanoparticle systems is Ascribed, as well as methods for such bio-programmable crystallization, and the products resultant from such methods. Specifically, the systems disclosed and taught herein are directed to improved strategies for the DNA-mediated self-assembly of multi-component functionalized nanoparticles into three-dimensional order surperlattices, wherein the functionalization of the nanoparticles with DNA is independent of either the composition of the material, or the shape of the nanoparticles.

### CROSS-REFERENCE TO A RELATED APPLICATION

This application claims the benefit under 35 U.S.C. 119(e) of U.S. Provisional Application No. 61/475,172 filed on Apr. 13, 2011, the content of which is incorporated herein in its entirety.

### STATEMENT OF GOVERNMENT LICENSE RIGHTS

This invention was made with Government support under contract number DE-AC02-98CH10886, awarded by the U.S. Department of Energy. The Government has certain rights in the invention.

### FIELD OF THE INVENTION

The inventions disclosed and taught herein relate generally to the field of DNA-mediated particle assembly, and, more specifically, to DNA-mediated self-assembly of multicomponent functionalized nanoparticles into three-dimensional (3D) ordered superlattices.

### BACKGROUND

The ability to control and regulate the kinetic behavior of DNA-based nanosystems is required for emerging nanoparticle applications in sensing, nano-device assembly, and gene delivery, among other applications. DNA-based methodology takes advantage of the tunable and programmable hybridization between DNA-capped nanomaterials. This approach has allowed for the development of sensitive detection systems based on the optical and physical properties of assembled nanoparticles, as well as detection based on their novel melting/disassembly properties.

In 1996, the Mirkin and Alivisatos groups showed that thiolated deoxyribonucleic acid (DNA) oligonucleotides can be attached onto gold nanoparticle surfaces to direct the formation of larger aggregations (Mirkin, C. A., et al., Nature, 1996. 382(65-92): p. 607-609; Alivisatos, A. P., et al., Nature, 1996. 382(6592): p. 609-611, each of which is incorporated by reference in its entirety). Since then, there have been many efforts to use the lock-and-key property of DNA to achieve ordered arrangements of gold nanoparticles. Only very recently, several groups independently demonstrated the successful DNA-guided three-dimensional crystallization of gold nanoparticles (Nykypanchuk, D., et al., Nature, 2008. 451(7178): p. 549-552; Park, S. Y., et al., Nature, 2008. 451(7178): p. 553-556; Xiong, H. M., D. van der Lelie, and O. Gang, Physical Review Letters, 2009. 102(1): p. 015504-(1-4); and Macfarlane, R. J., et al., Angewandie Chemie-International Edition, 2010. 49(27): p. 4589-4592, each of which is incorporated herein by reference in its entirety). In these studies, it was found that either face-centered cubic (FCC) or body-centered cubic (BCC) structures with tunable lattice parameters can be formed by controlling the type, number and length of the DNA sequences. DNA length, rigidity, and number were proven to be the key parameters for gold nanoparticle crystallization.

During the last decade, functional nanomaterials have become a hot research topic due to their importance and wide-spread application potential, ranging from magnetic recording media, catalysts, solar cells, biomedicine, and so on. The ability to assemble multi-component nanoparticles into three-dimensional ordered superstructures is of particular interest for building advanced metamaterials with novel magnetic, plasmonic, photonic, and catalytic properties. Among many assembly techniques, DNA-mediated nanoparticle assembly has emerged as a powerful and versatile strategy that has many advantages due to the synthetically programmable length and recognition properties of DNA.

However, up to now, assembly in organized structures of DNA-functionalized objects has mainly been limited to gold nanoparticles. The main reason being that gold nanoparticles can be easily coated with a dense DNA shell by simply replacing the weak surfactants. e.g., citrate, cetyltrimethylammonium bromide (CTAB) etc. used during the synthesis process, by thiolated DNA. For synthesis of nanoparticles with different composition other than gold or gold materials with more complex morphologies, the functionalization is very difficult because surfactants that are routinely used with these nanoparticles bind tightly to the surface, making their removal very difficult. For instance for Au nanoparticles with complex shapes, such as Au rhombic dodecahedra and octahedra with cetylpyridinium chloride (CPC) as surfactant, directly replacing CPC with thiolated DNA will result in Au particle aggregation due to the low DNA-CPC exchange efficiency. In this case, fortunately, since CPC is not a very strong surfactant, one can first use a high CTAB concentration to partially exchange CPC, and then replace CTAB by thiolated DNA (Jones. M. R., et al., Nature Materials, 2010. 9(11): p. 913-917, which is incorporated herein by reference in its entirety).

However, for Au polyhedrons synthesized with much stronger ligands or long polymers as surfactants, like poly-diallyl-dimethylammonium chloride (PDDA) and poly-vinyl-pyrrolidone (PVP), the surfactants are very difficult to replace and consequently, to date, there are no reports on their functionalization with DNA and use in programmable assemblies. For materials other than gold, such as palladium nanoparticles synthesized with PVP, direct thiolated DNA functionalization is impossible due to difficulty to DNA penetration and the much weaker thiol-palladium affinity. As a result of these functionalization problems, the components for DNA directed ordered nanoparticle assembly and crystallization have been limited to gold. Additionally, although there have been some recent reports on extending the particle component to other inorganic materials, such as silver (Lee, J. S., et al., Nano Letters, 2007. 7(7): p. 2112-2115; Pal, S., et al., Chemical Communications. 2009(40): p. 6059-6061, each of which is incorporated herein by reference in its entirety), quantum dots (Maye, M. M., et al., Chemical Communications, 2010. 46(33): p. 6111-6113, which is incorporated herein by reference in its entirety), silica (Hilliard, L. R., et al., Analytica Chimica Acta, 2002. 470(1): p. 51-56, which is incorporated herein by reference in its entirety) and iron oxides (Cutler, J. L., et al., Nano Letters, 2010. 10(4): p. 1477-1480; Lee, C. W., et al., Journal of Magnetism and Magnetic Materials. 2006. 304(I): p. E412-E414, each of which is incorporated herein by reference in its entirety), there are still no reports on incorporating such materials into three-dimensional (3D) ordered structures using the concept of programmable assembly offered by functionalization with biological compounds, including nucleic acids, preferably DNA, and proteins.

For most types of particles used for catalysis and other advanced applications, surface capping with high affinity ligands or long polymers is inevitable during their synthesis process. This makes it hard for DNA to replace or penetrate the ligand shell, and thus functionalization becomes a challenge. Furthermore, the application of strong ligands is not only limited to nano particles with composition different from gold, for instance quantum dots (QD) (Murray, C. B., et al., Journal of the American Chemical Society, 1993. 115(19): p. 8706-8715; Dabbousi, B. O., et al., Journal of Physical Chemistry B, 1997. 101(46): p. 9463-9475, each of which is incorporated herein by reference in its entirety) or palladium (Lim, B., et al., Advanced Functional Materials, 2009 19(2): p. 189-200, which is incorporated herein by reference in its entirety), but also to synthesize and preserve the shapes of non-spherical particles, even for Au (Sun, Y. G. and Y. N. Xia, Science, 2002. 296(5601): p. 2176-2179, which is incorporated herein by reference in its entirety).

In sum, several challenges remain for the full exploitation of DNA-mediated assembly of heterogeneous nanoparticle assembly. DNA-functionalized nano objects are mainly limited to gold nanoparticles. Materials coated with high affinity ligands or polymers, such as palladium nanoparticles coated with PVP, or gold nanoparticles coated with PVP or PDDA, fail to be further functionalizable with biological molecules with the current state of the art. The range of nano objects successfully used for DNA-directed crystallization has been limited to gold nanoparticles. Although there are a few, limited reports of DNA functionalized nanoparticle other than gold, such as silver, quantum dots, silicon and iron oxides, these nanoparticles have never been exploited as nanoparticle building blocks that were subsequently used for the programmable assembly of 3D artificial materials. The structures of DNA-guided nanoparticle-nanoparticle assemblies have so far been limited to body-centered cubic (BCC) and face-centered cubic (FCC) structures, which compromises novel structure-related properties and their advanced applications. Additionally, the cost of using thiolated DNA for gold nanoparticle functionalization is very high compared to using biotinylated DNA.

### SUMMARY

The present disclosure describes a general strategy for DNA-mediated self-assembly of multicomponent functionalized nanoparticles into three-dimensional (3D) ordered superlattices. The generally applicable strategy either allows for removal of the high affinity ligands that bind to the nanoparticle surface and their replacement with other ligands that do allow for subsequent functionalization with biological groups (mostly for hydrophilic nanoparticles), or provision of an additional ligand layer that allows for further functionalization with biological groups (mostly for hydrophobic nanoparticles), which can prevent irreversible and uncontrolled aggregation of nanoparticles while preserving their unique structures and physical properties. Such nanomaterials can then be applied in various programmable assembly strategies.

The disclosure also demonstrates a generally applicable strategy of how to functionalize nanoparticles with DNA, independent of the composition of the material or the shape of the nanoparticles. The generally applicable strategy includes three steps, namely, carboxylic group grafting, streptavidin (STV)-conjugation, and biotinylated-DNA attachment. In the first step, the ligands having a carboxylic group are adopted for the nanoparticles by replacing the original high affinity ligands or providing additional ligands with the carboxylic acid functional groups. In particular, short mercapto acid ligands, such as mercaptoundecanoic acid, and amphiphilic polymers, such as lipid-PEG carboxylic acid, may be used.

The subsequent two steps rely on 1-ethyl-3-[3-dimethylaminopropyl]-carbodiimide hydrochloride (EDC)-assisted chemistry and high specific and strong STV-biotin binding. This DNA functionalization strategy is very versatile and can be applied to a broad range of functional nanoparticles. In the EDC-assisted streptavidin (STV)-conjugation the conjugate streptavidin can be covalently bound to the particle surface by a reaction between the carboxyl (COOH) group of the ligand and the amine (NH₂) groups abundant on the streptavidin (STV) surface. Finally, biotinylated-DNA is coupled with STV on the particle surface due to the specific binding between biotin and STV. This strategy has been successfully demonstrated to assemble organized superstructures with magnetic (Fe₂O₃), plasmonic (Au), photonic (quantum dot), and catalytic (Pd) materials, and protein (such as STV), as well as combinations thereof. Also demonstrated is that these ordered structures possess rich phases that until now could not be obtained using the current state of the art in nanomaterial assembly approaches.

### BRIEF DESCRIPTION OF THE DRAWINGS

The following Figures form part of the present specification and are included to further demonstrate certain aspects of the present invention. The invention may be better understood by reference to one or more of these Figures in combination with the detailed description of specific embodiments presented herein.

FIG. 1A shows SEM and TEM images for Pd nano-octahedra (NO).

FIG. 1B shows SEM and TEM images for Pd nanocubes (NC).

FIG. 1C shows SEM and TEM images for Pd nanododecahedra (ND).

FIG. 2A shows TEM images of Pd NCs with an edge size of 6±0.5 nm.

FIG. 2B is a TEM image of Pd NCs with an edge size of 10±0.8 nm.

FIG. 2C is a TEM image of Pd NCs with an edge size of 12±0.9 nm.

FIG. 2D shows TEM images of Pd NCs with an edge size of 23±2.6 nm.

FIG. 2E shows TEM images for Pd NOs with an edge size of 15±1.3 nm.

FIG. 3 is a schematic illustration of the assembly system for direct hybridization of binary nanoparticles or nanoparticles and protein entities.

FIG. 4A is a TEM image of thiol-DNA capped Au nanoparticles with a diameter of 6.2±1 nm.

FIG. 4B is a TEM image of thiol-DNA capped Au nanoparticles with a diameter of 8.8±1.7 nm.

FIG. 4C is a TEM image of thiol-DNA capped Au nanoparticles with a diameter of 12.5±1.8 nm.

FIG. 4D is a TEM image of thiol-DNA capped Au nanoparticles with a diameter of 14.7±2 nm.

FIG. 4E is a TEM image of streptavidin (STV)-capped Au nanoparticles with diameter of 16.6±1.5 nm.

FIG. 5A illustrates a 2D SAXS pattern and its corresponding S(q) of the Sys-AA9₁₈.

FIG. 5B illustrates a 2D SAXS pattern and its corresponding S(q) of the Sys-AA9₃₀.

FIG. 5C illustrates a 2D SAXS pattern and its corresponding S(q) of the Sys-AA9₅₀.

FIG. 5D illustrates a 2D SAXS pattern and its corresponding S(q) of the Sys-AA9₈₀.

FIG. 5E illustrates a 2D SAXS pattern and its corresponding S(q) of the Sys-AA6₅₀.

FIG. 5F illustrates a 2D SAXS pattern and its corresponding S(q) of the Sys-AA12₅₀.

FIG. 5G illustrates a 2D SAXS pattern and its corresponding S(q) of the Sys-AA15₅₀.

FIG. 5H illustrates a 2D SAXS pattern of the melting Sys-AA9₅₀ at 710° C., with the gray and black 1D curves corresponding to the scattering intensity of melting and assembled Sys-AA9₉₀, respectively.

FIG. 5I illustrates fitting of the melting curve of Sys-AA9₅₀.

FIG. 5J illustrates a 2D SAXS pattern of the melting Sys-AA15₅₀ and its fitting.

FIG. 6A illustrates an exemplary schematic of the Cu₃Au structure (left) and the calculated S(q) for this structure using Powder Cell in a two-atom system with an atom number ratio (AR) of 17.

FIG. 6B illustrates the calculated S(q) for the Cu₃Au structure with an AR of 5.

FIG. 6C illustrates a schematic of the NaTl structure (left) and the calculated S(q) for this structure with an AR of 17.

FIG. 6D illustrates the calculated S(q) for the NaTl structure with an AR of 5.

FIG. 6E illustrates the calculated S q) for the NaTl structure with an AR of 2.

FIG. 6F illustrates the calculated S(q) for the NaTl structure with an AR of 1.5.

FIG. 7A illustrates the 2D SAXS pattern and corresponding S(q) for the Sys-POA.

FIG. 7B illustrates the 2D SAXS pattern and corresponding S(q) for the Sys-PCA.

FIG. 7C illustrates the 2D SAXS pattern and corresponding S(q) for the Sys-PDA₁₈.

FIG. 7D illustrates the 2D SAXS pattern and corresponding S(q) for the Sys-PDA₃₀.

FIG. 7E illustrates the 2D SAXS pattern and corresponding S(q) for the Sys-PDA₅₀.

FIG. 7F illustrates the 2D SAXS pattern and corresponding S(q) for the Sys-PDA₈₀.

FIG. 7G illustrates the 2D SAXS pattern and corresponding Ip(q) for a system of Pd NDs and Au without a linker.

FIG. 7H illustrates the 2D SAXS pattern and corresponding S(q) for Sys-PDA₅₀ at 710° C. (black curve), and after cooling down (gray curve).

FIG. 8A shows a TEM image of Q705, where the QD has elongated shape, and the size distribution histogram of long axis length and short axis length of the QDs in the image.

FIG. 8B illustrates Ip(q), the fitting, and size distribution for Q705.

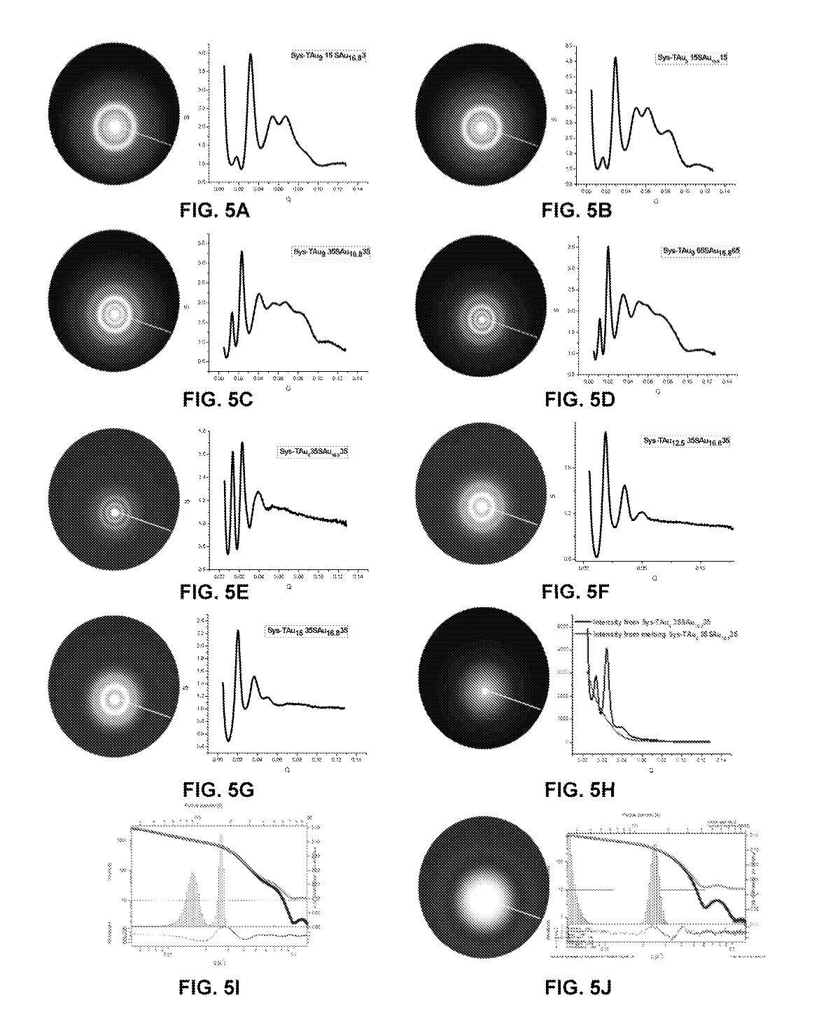

FIG. 5C illustrates Ip(q), the fitting, and size distribution for Q605.

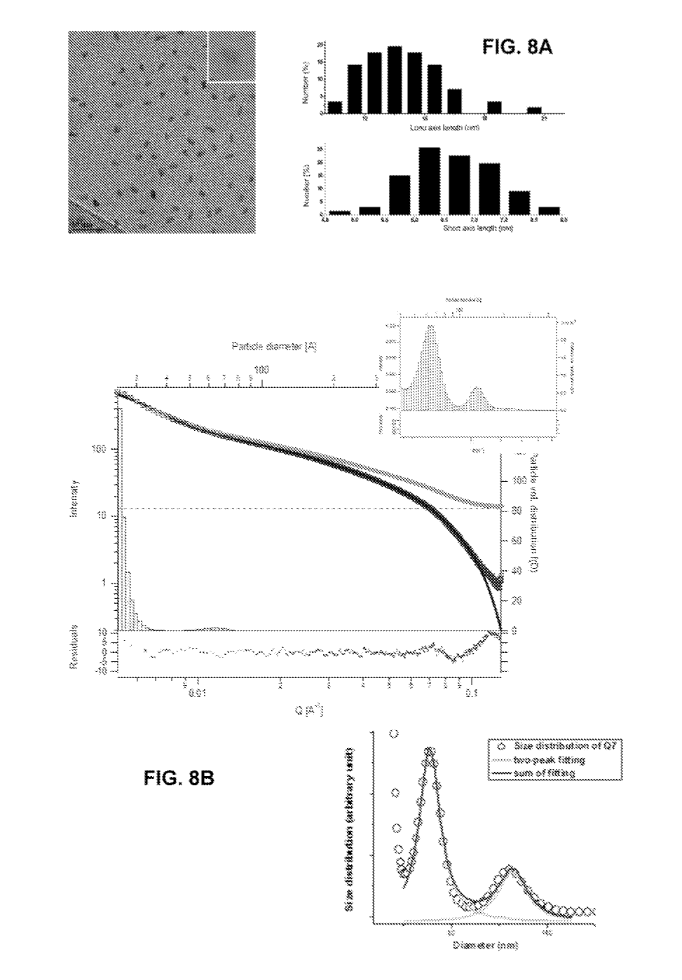

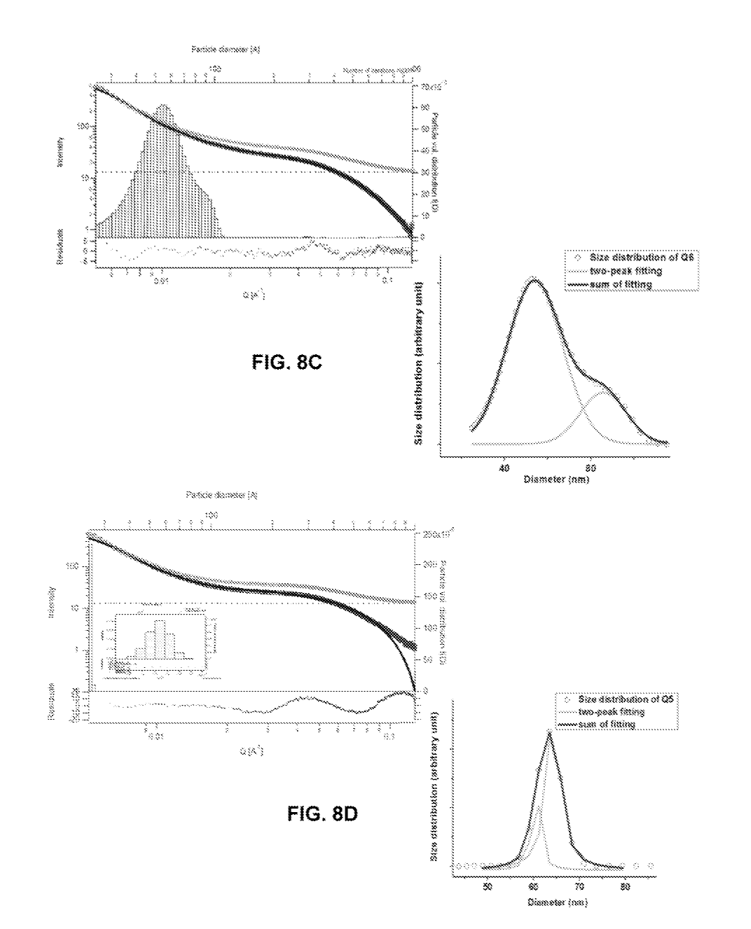

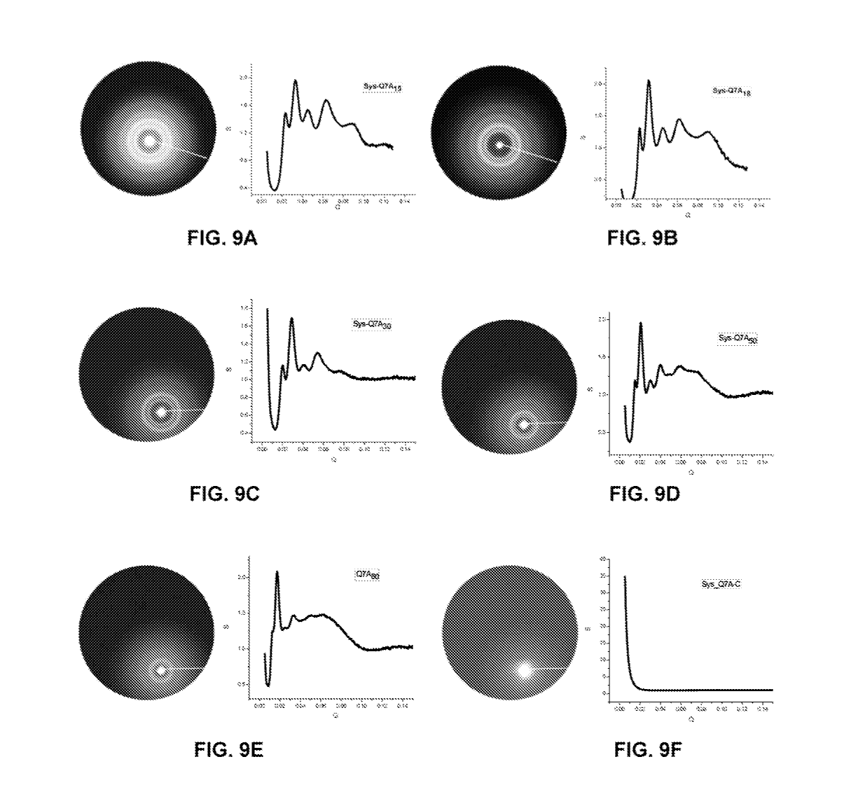

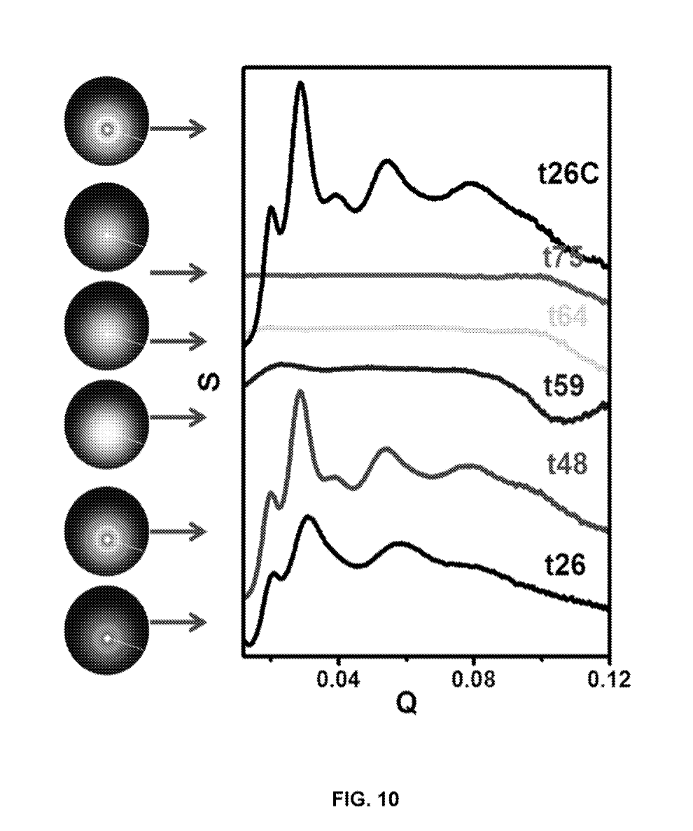

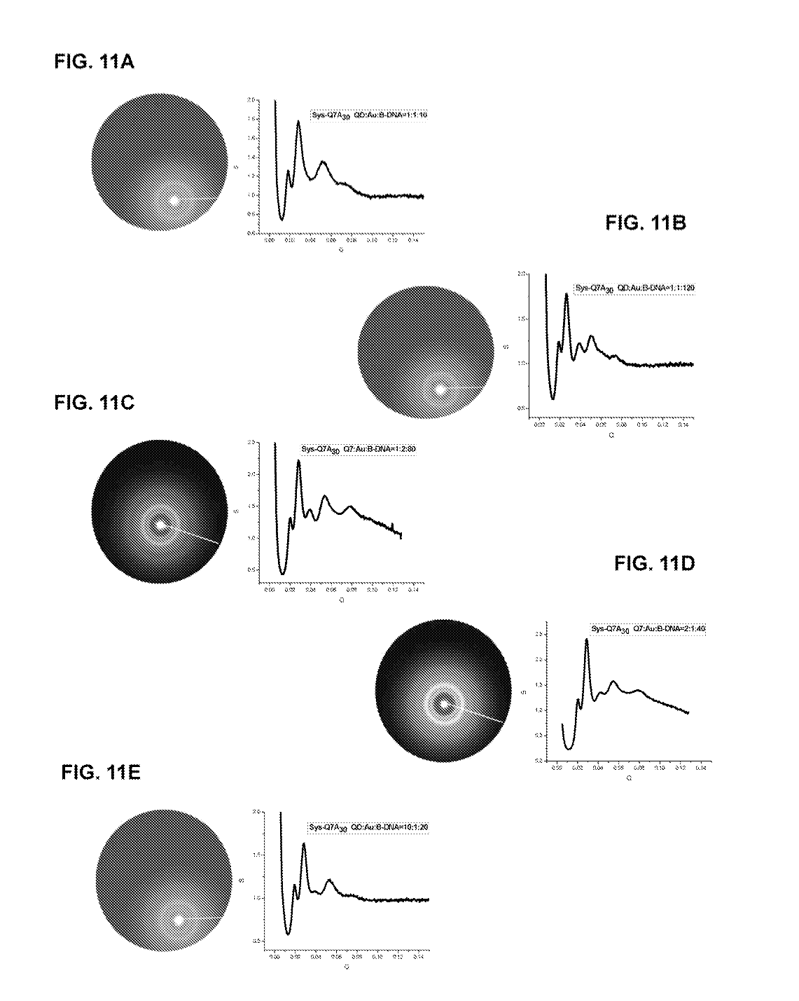

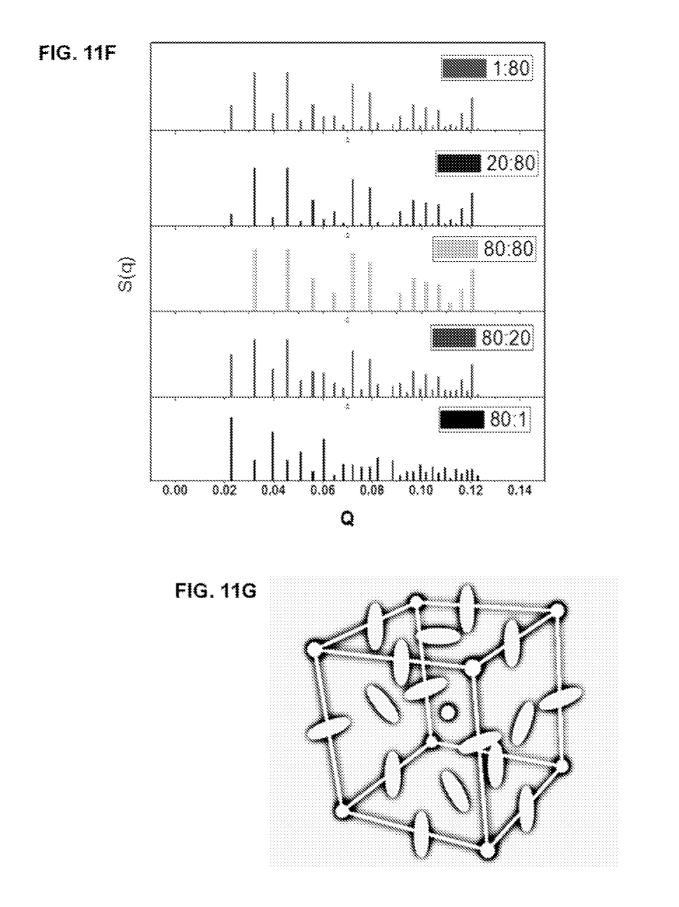

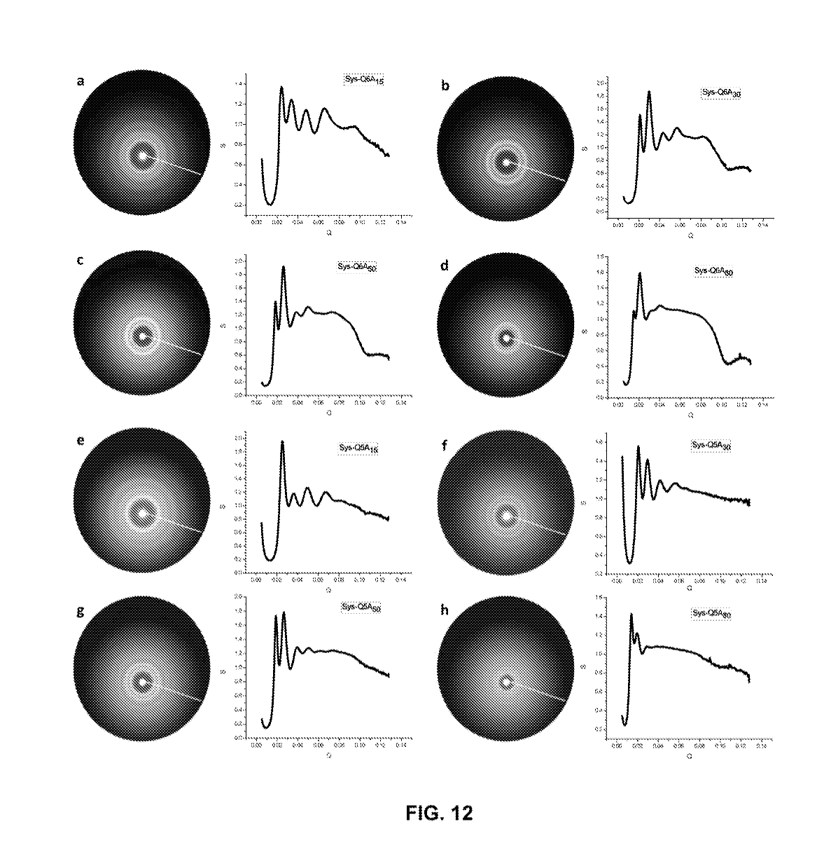

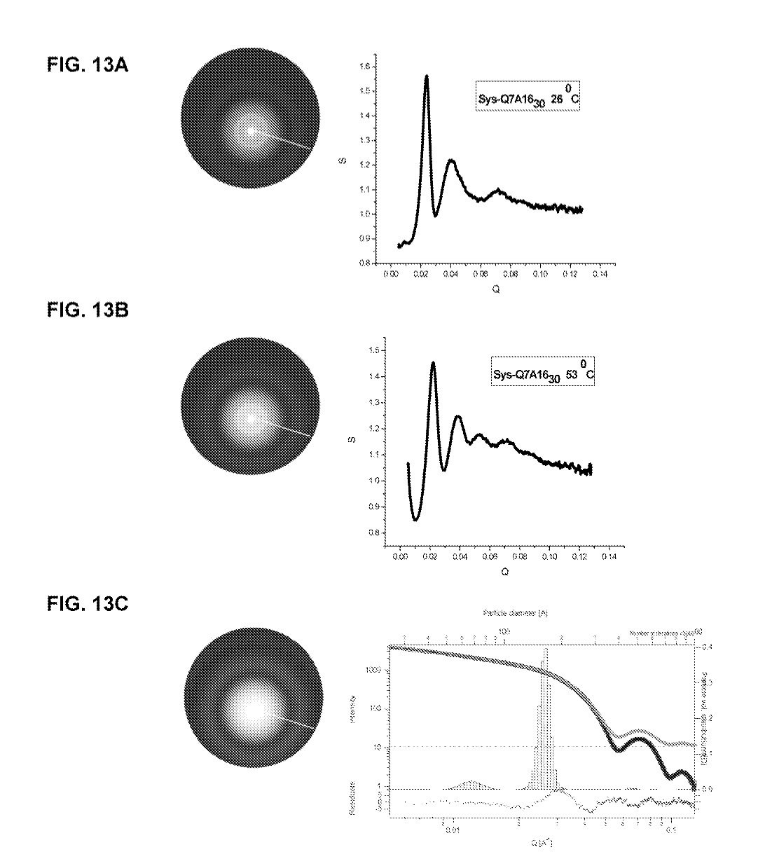

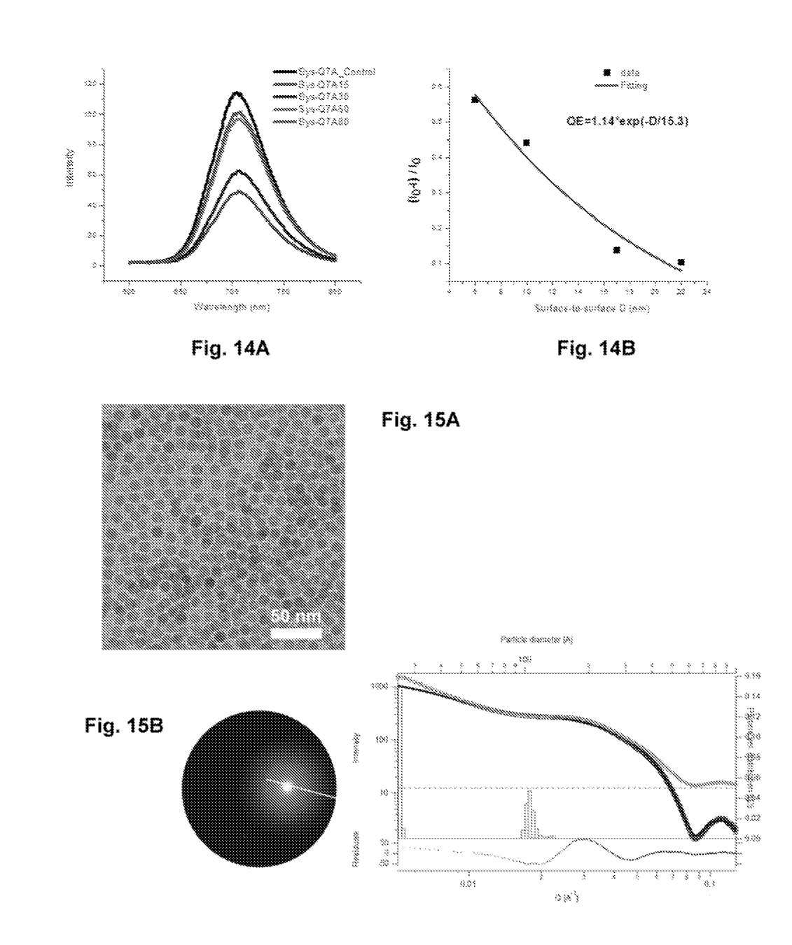

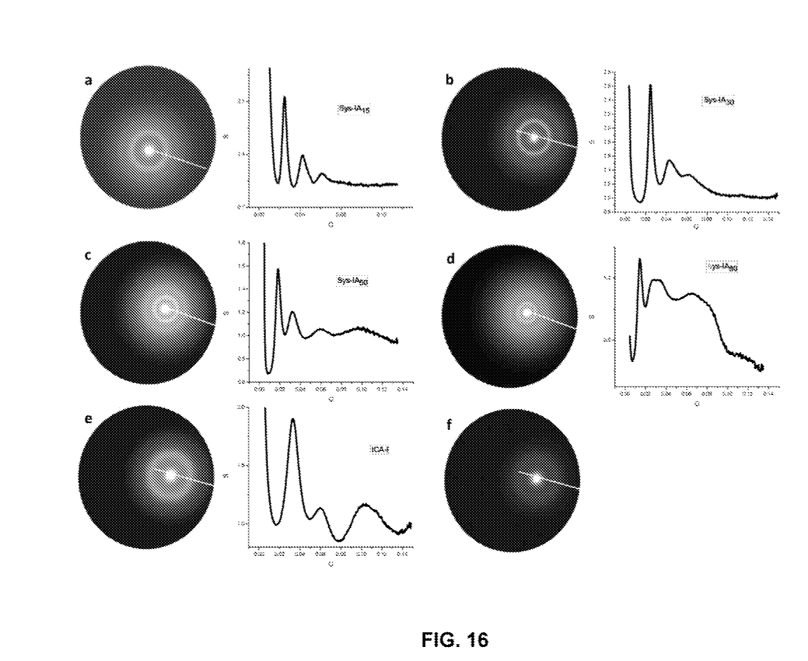

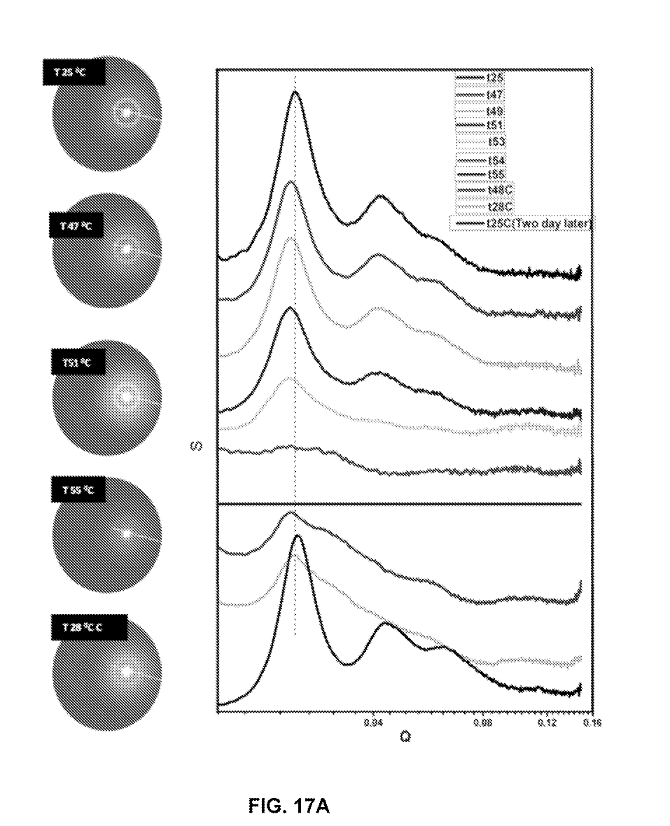

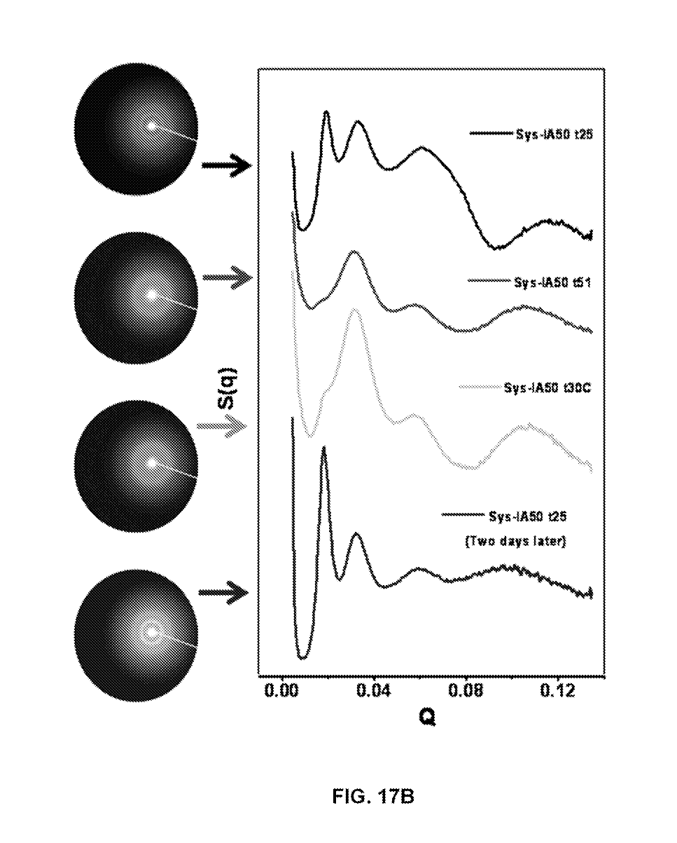

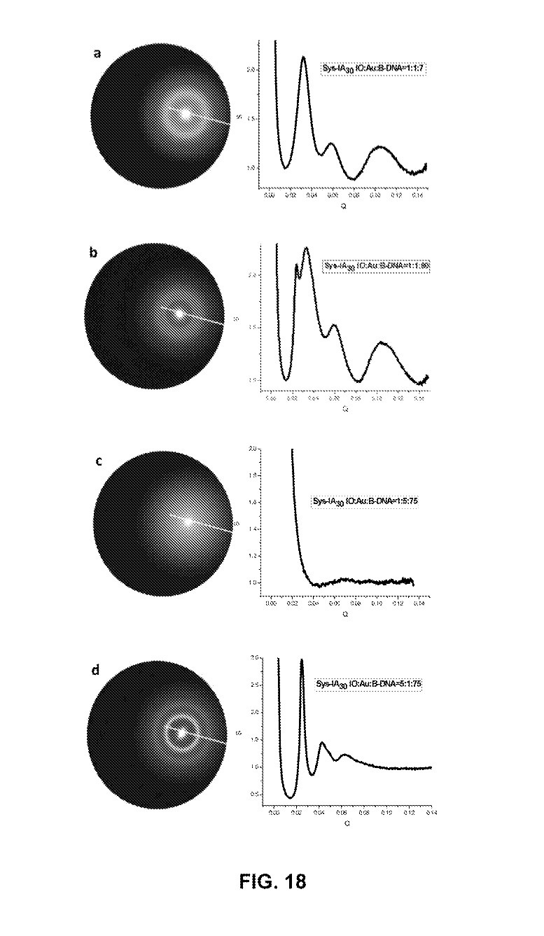

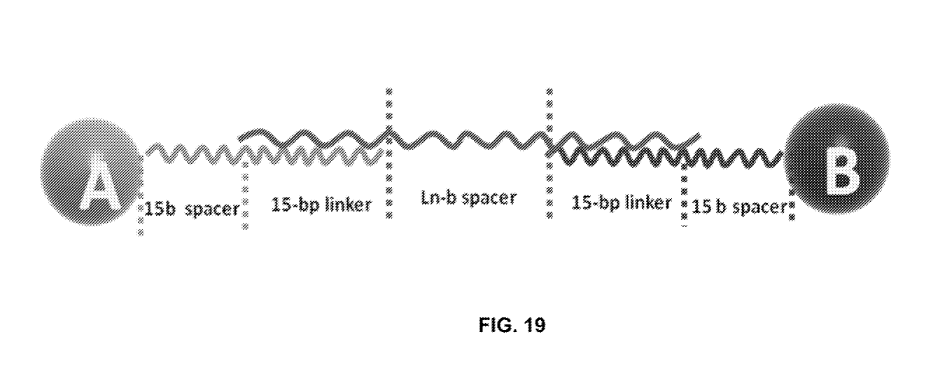

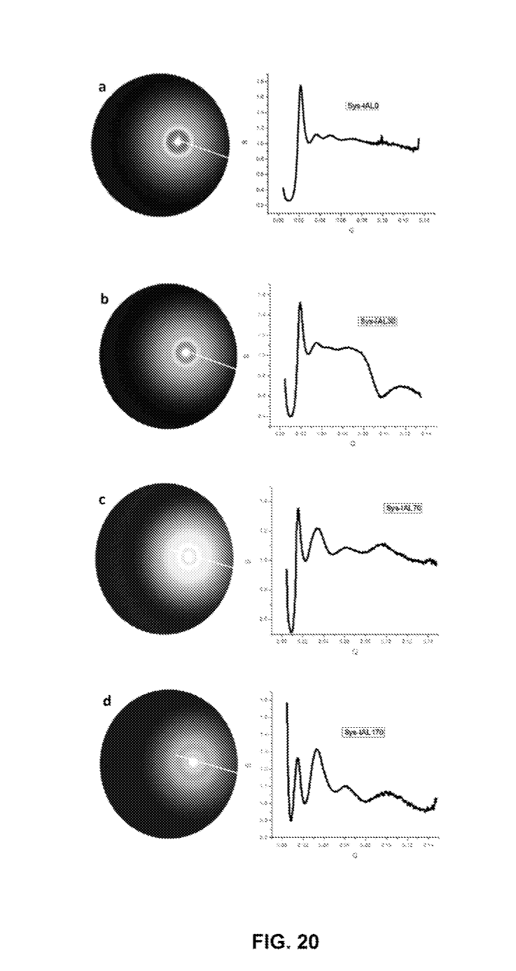

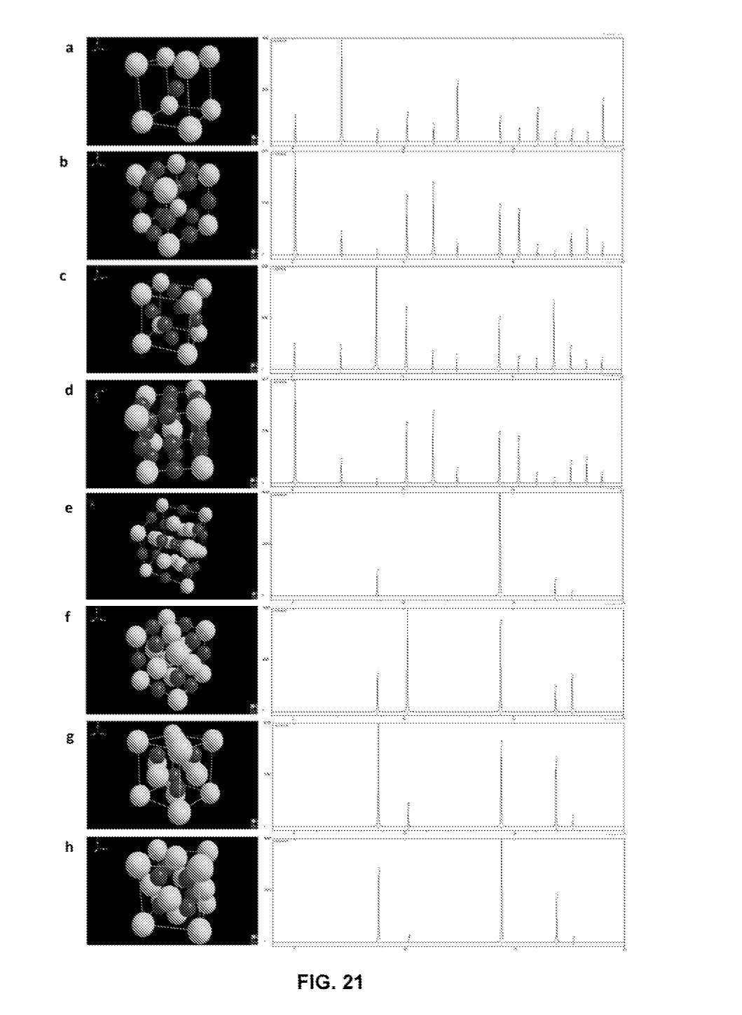

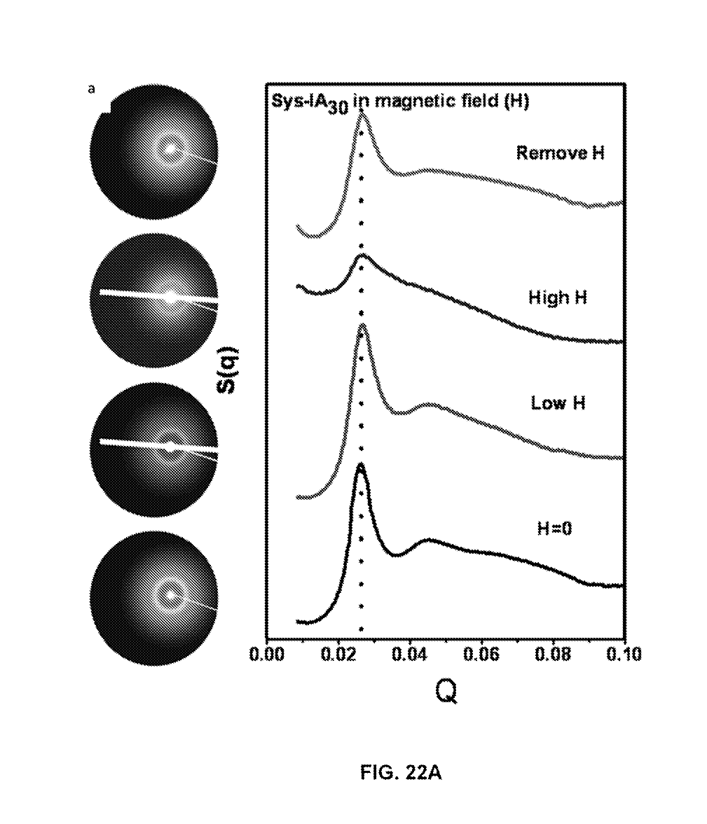

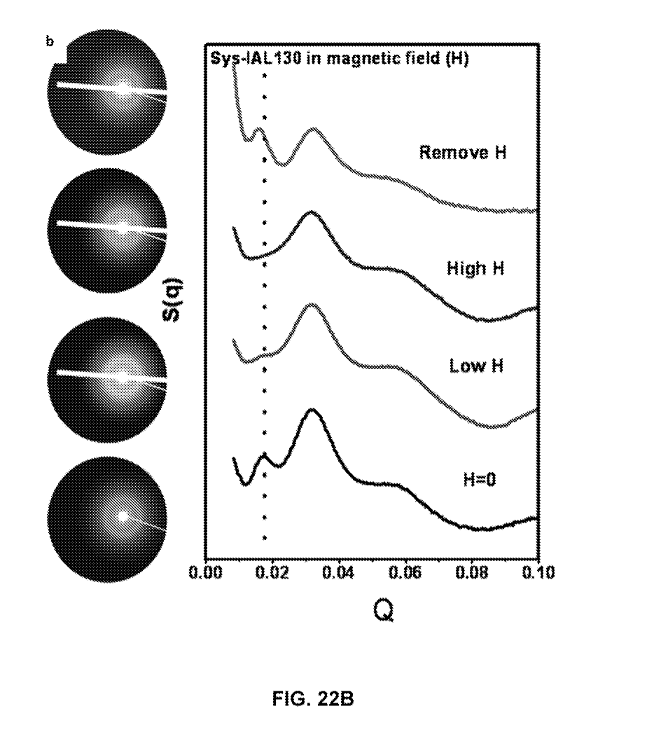

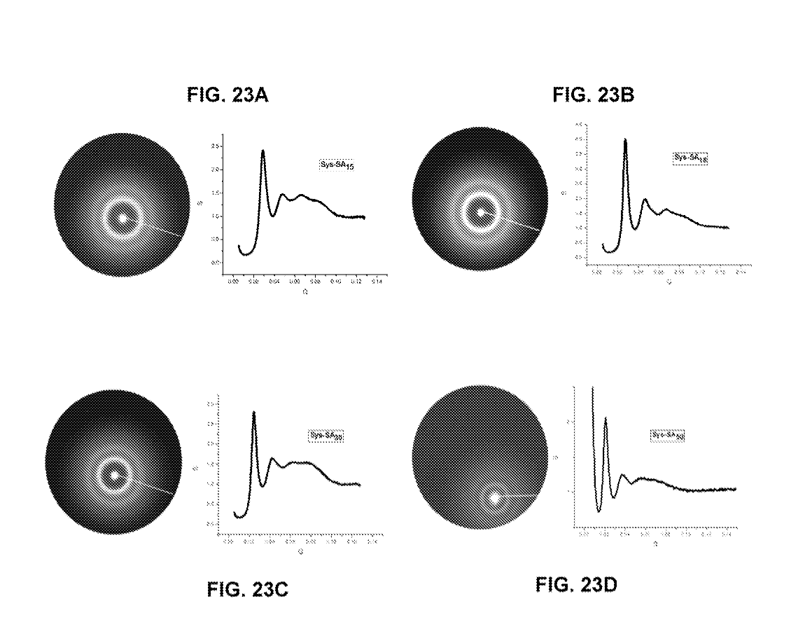

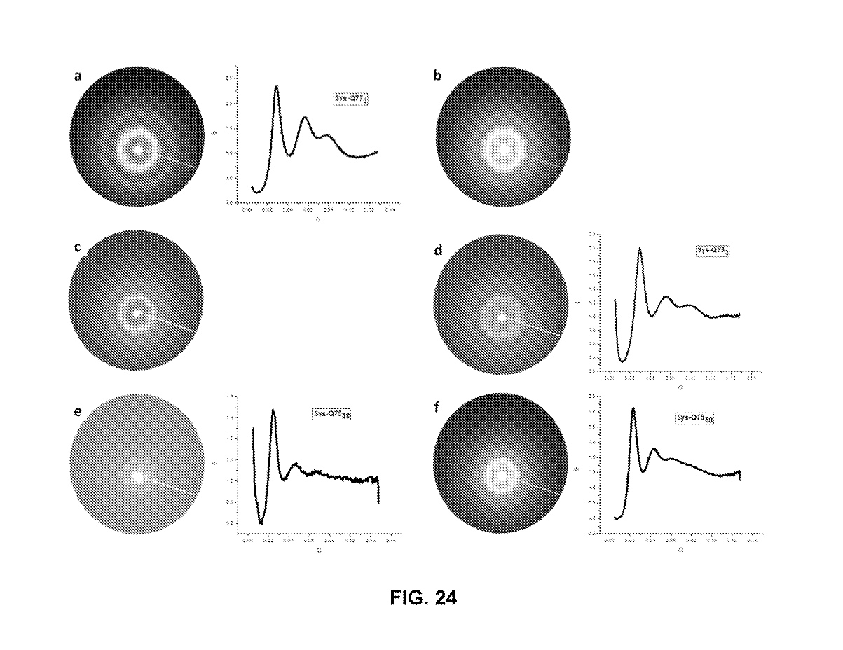

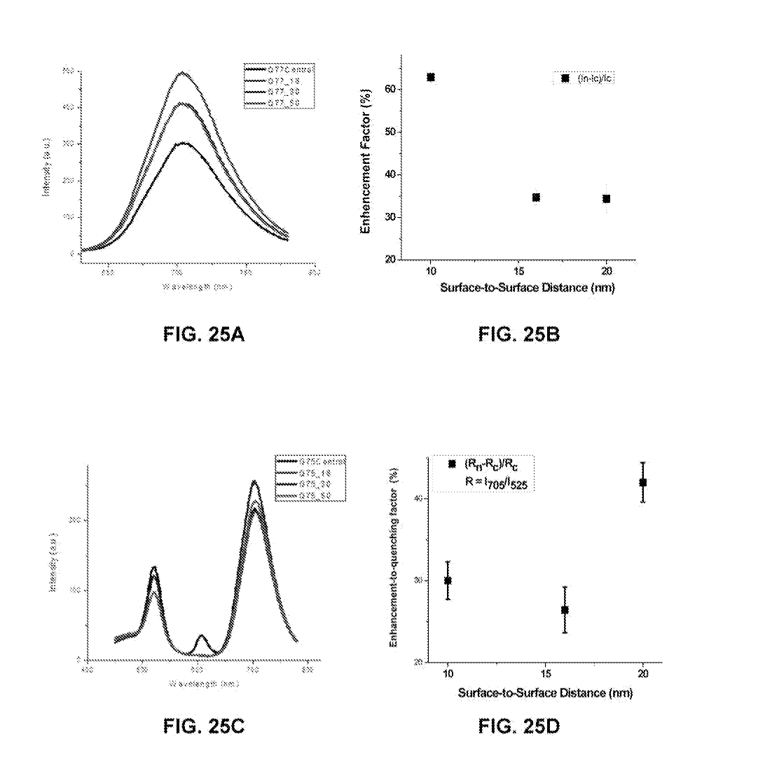

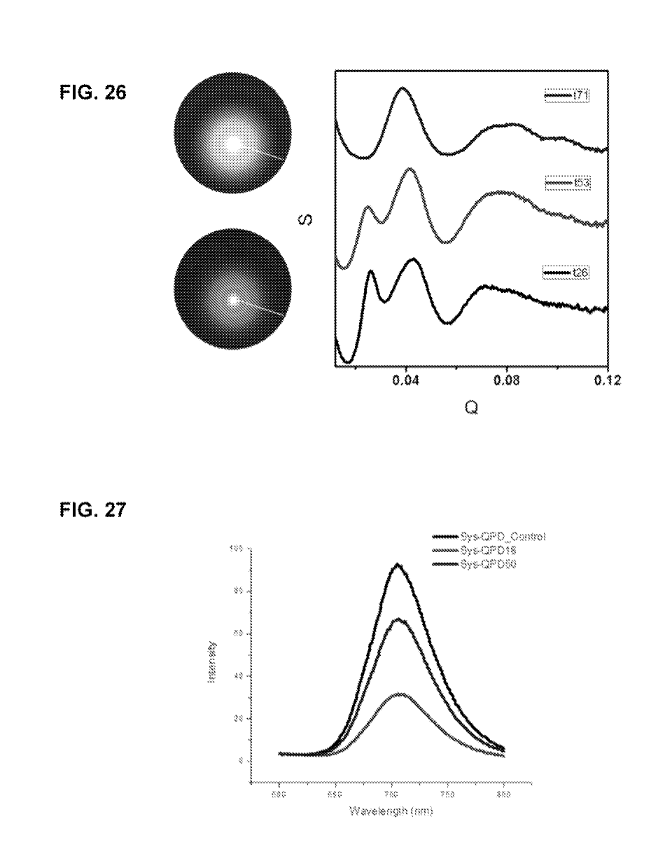

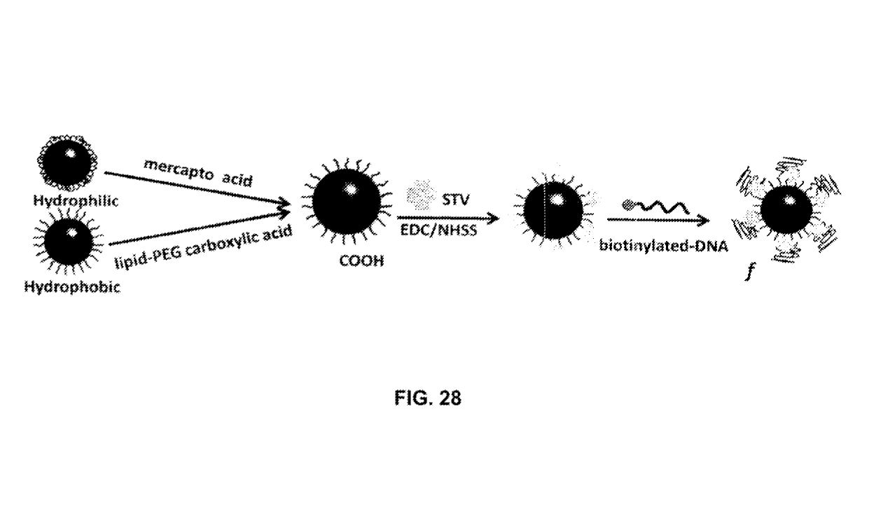

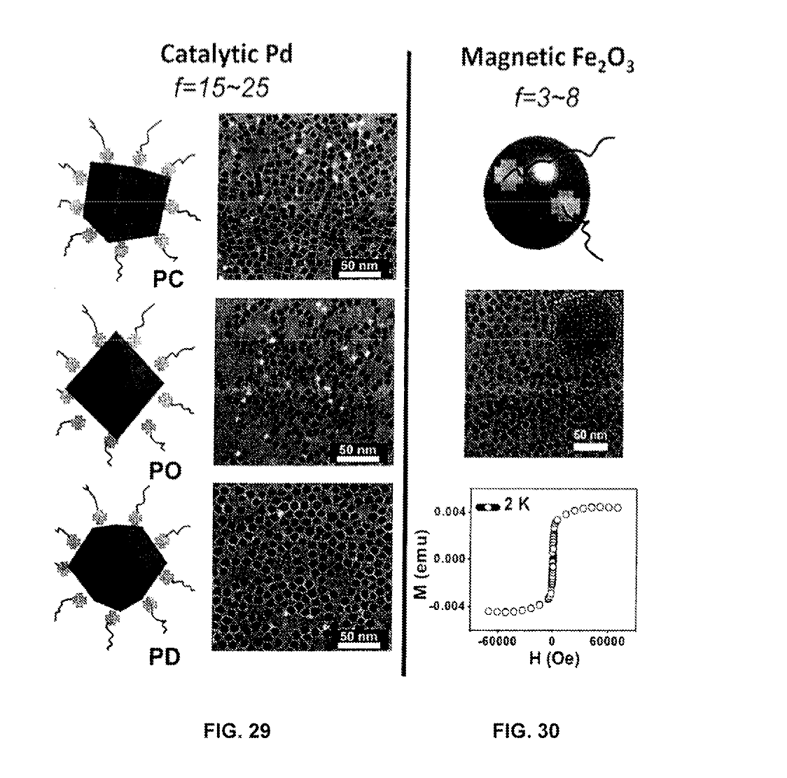

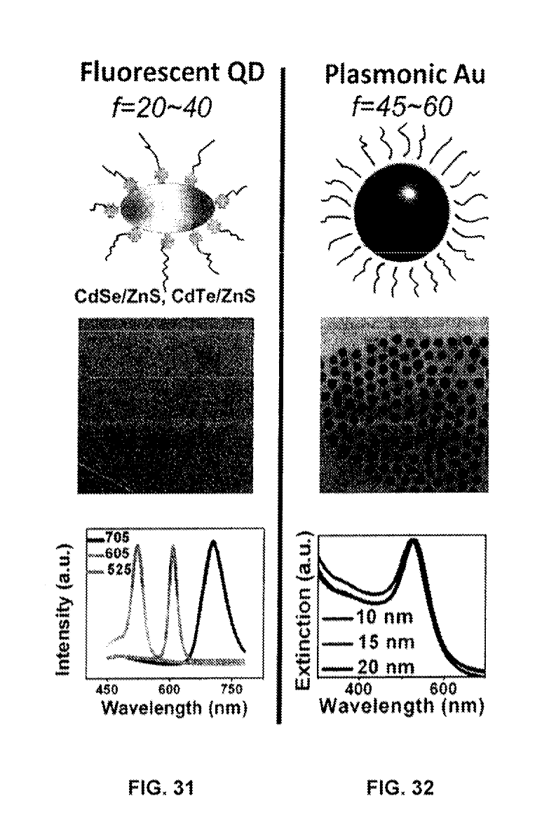

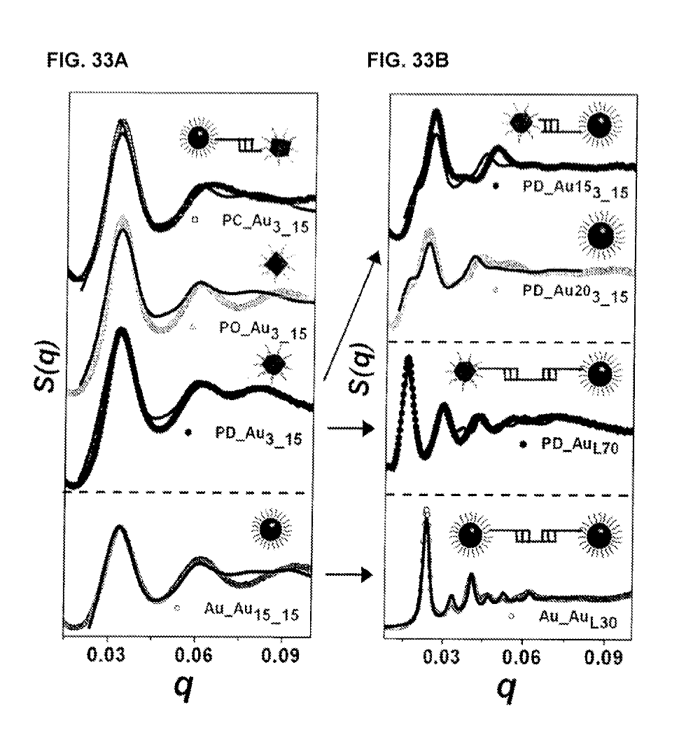

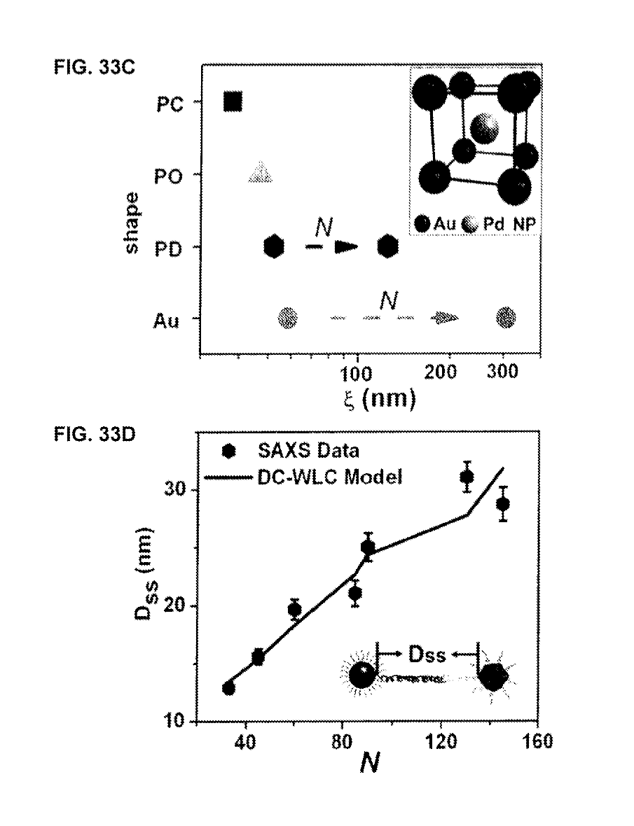

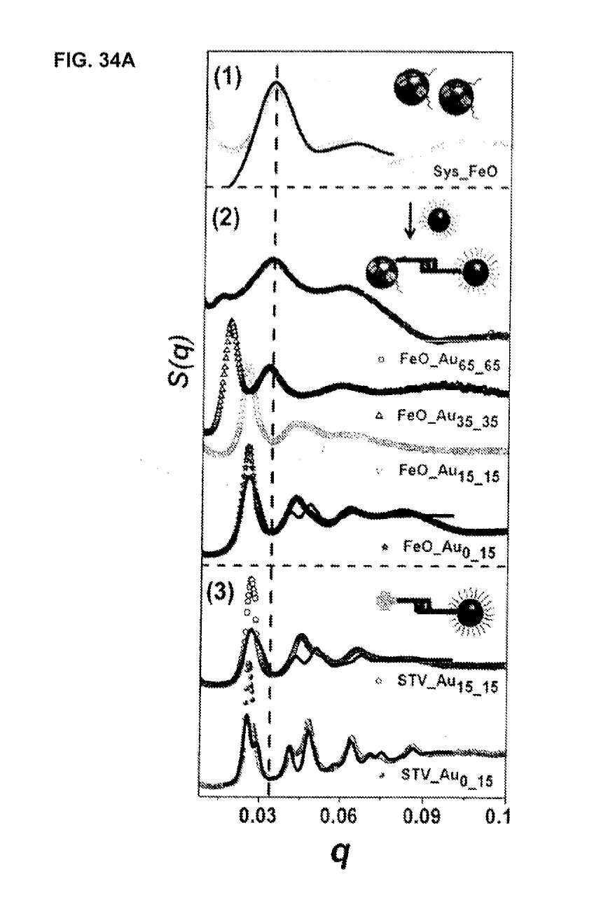

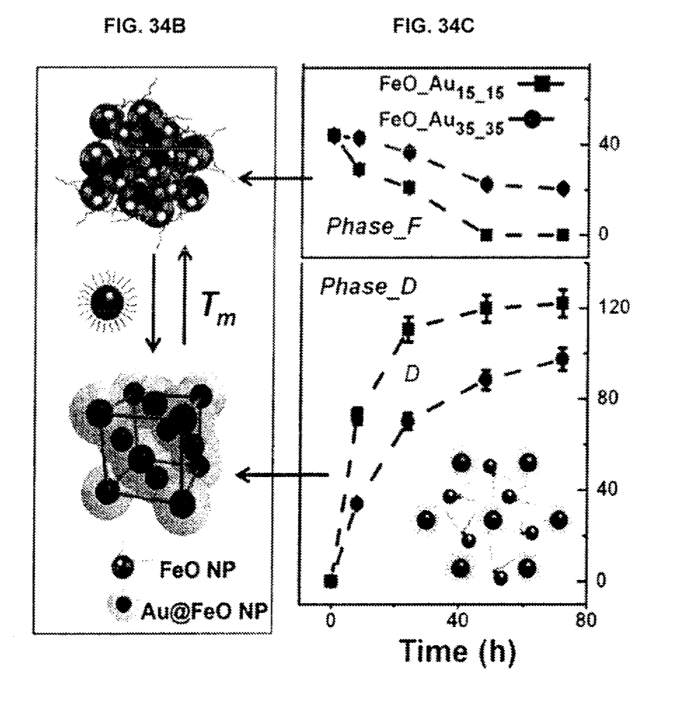

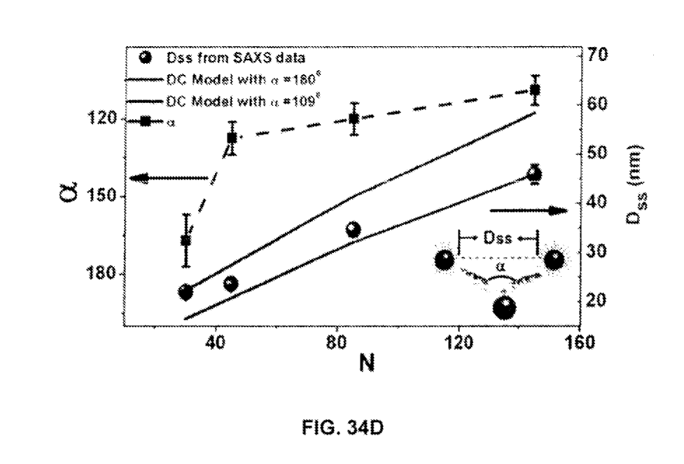

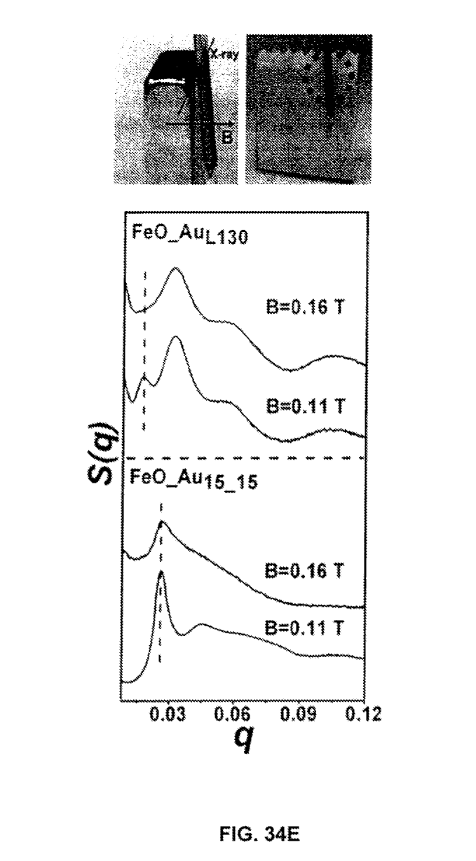

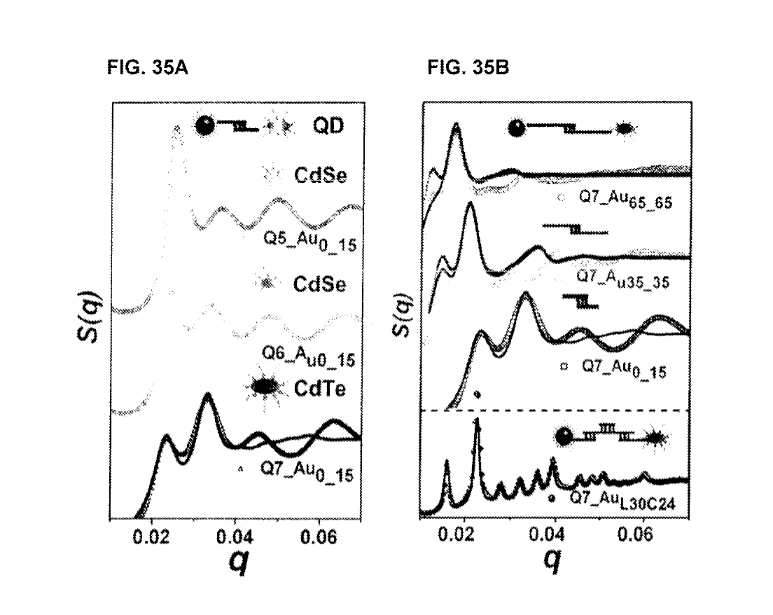

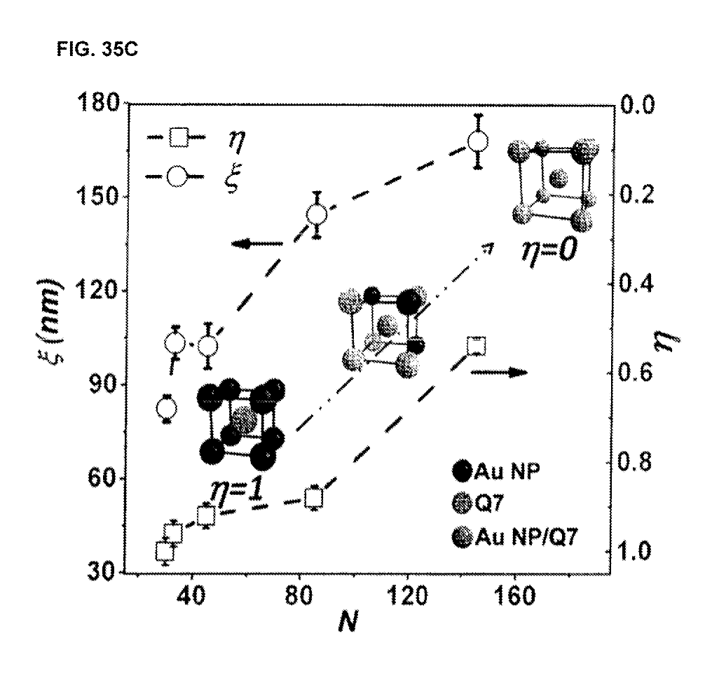

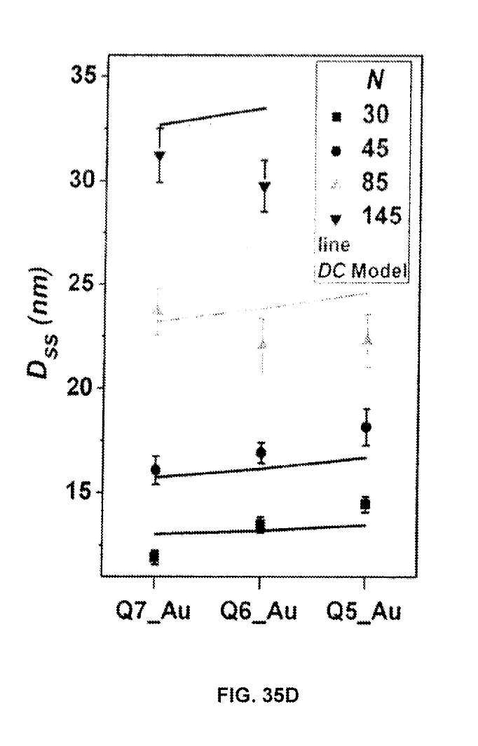

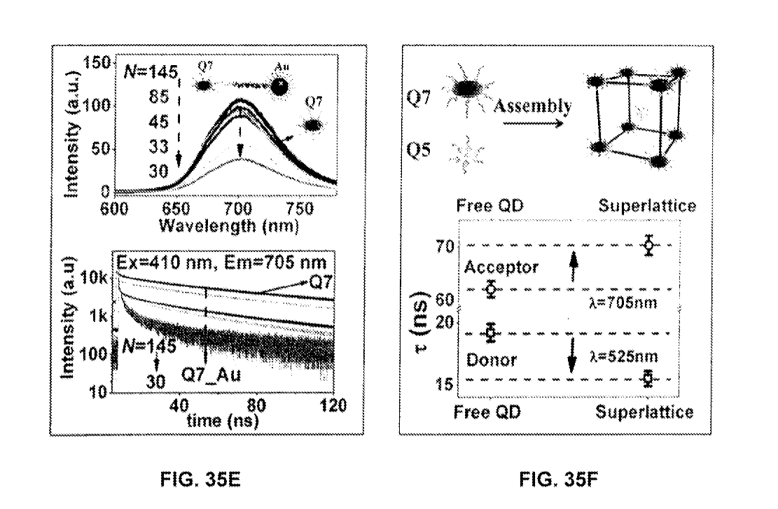

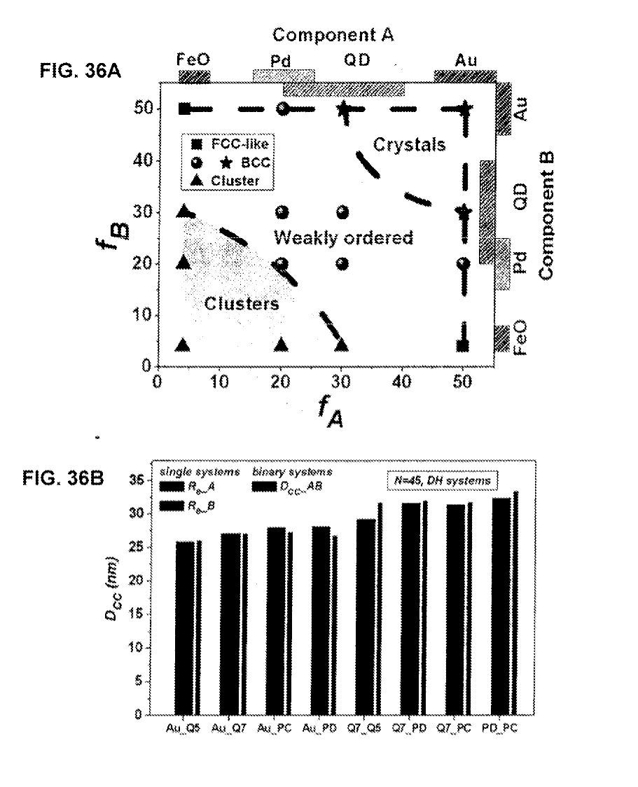

FIG. 8D illustrates Ip(q), the fitting, and size distribution for Q525.

FIG. 9A illustrates the 2D SAXS pattern and corresponding S(q) for Sys-Q7Aₙ for n=15.

FIG. 9B illustrates the 2D SAXS pattern and corresponding S(q) for Sys-Q7Aₙ for n=18.

FIG. 9C illustrates the 2D SAXS pattern and corresponding S(q) for Sys-Q7Aₙ for n=30.

FIG. 9D illustrates the 2D SAXS pattern and corresponding S(q) for Sys-Q7Aₙ for n=50.

FIG. 9E illustrates the 2D SAXS pattern and corresponding S(q) for Sys-Q7Aₙ for n=80.

FIG. 9F illustrates the 2D SAXS pattern and corresponding Ip(q) for a system of Q705 and Au without a linker.

FIG. 10 illustrates the temperature-dependent phase behavior for Sys-Q7A₃₀ without pre-annealing.

FIG. 11A illustrates the 2D SAXS pattern and corresponding S(q) for Sys-Q7A₃₀ with a mole ratio of QD:Au:Biotin-DNA::1:1:10.

FIG. 11B illustrates the 2D SAXS pattern and corresponding S(q) for Sys-Q7A₃₀ with a mole ratio of QD:Au:Biotin-DNA::1:1:120.

FIG. 11C illustrates the 2D SAXS pattern and corresponding S(q) for Sys-Q7A₃₀ with a mole ratio of QD:Au:Biotin-DNA::1:2:80.

FIG. 11D illustrates the 2D SAXS pattern and corresponding S(q) for Sys-Q7A₃₀ with a mole ratio of QD:Au:Biotin-DNA::2:1:40.

FIG. 11E illustrates the 2D SAXS pattern and corresponding S(q) for Sys-Q7A₃₀ with a mole ratio of QD:Au:Biotin-DNA::10:1:20.

FIG. 11F illustrates a schematic of the La₂O₃ structure and the calculated S(q) for this structure using Powder Cell in a two-atom system with atom number ratio (AR) labeled in the figure.

FIG. 12A illustrates the 2D SAXS pattern and corresponding S(q) for Sys-QA6ₙ with n=15.

FIG. 12B illustrates the 2D SAXS pattern and corresponding S(q) for Sys-QA6ₙ with n=30.

FIG. 12C illustrates the 2D SAXS pattern and corresponding S(q) for Sys-QA6ₙ with n=50.

FIG. 12D illustrates the 2D SAXS pattern and corresponding S(q) for Sys-QA6ₙ with n=80.

FIG. 12E illustrates the 2D SAXS pattern and corresponding S(q) for Sys-QA5ₙ with n=15.

FIG. 12F illustrates the 2D SAXS pattern and corresponding S(q) for Sys-QA5ₙ with n=30.

FIG. 12G illustrates the 2D SAXS pattern and corresponding S(q) for Sys-QA5ₙ with n=50.

FIG. 12H illustrates the 2D SAXS pattern and corresponding S(q) for Sys-QA5ₙ with n=80.

FIG. 13A illustrates the 2D SAXS pattern and corresponding S(q) Sys-Q7A16₃₀ at 260° C.

FIG. 13B illustrates the 2D SAXS pattern and corresponding S(q) Sys-Q7A16₃₀ at 530° C.

FIG. 13C illustrates the 2D SAXS pattern, its Ip(q), and the fitting at 710° C. for the melting system.

FIG. 14A illustrates the photoluminescence of Sys-Q7A.

FIG. 14B illustrates a plot of the quenching efficiency of Sys-Q7A against the surface-to-surface distance between the QD and Au obtained by SAXS. The solid line is a fitting using an exponential decay model.

FIG. 15A illustrates a TEM image of iron oxide Fe₂O₃ (also referred to as IO or FeO) nanoparticles.

FIG. 15B illustrates the SAXS Ip(q) and the fitting for the IO nanoparticles, which indicate that they have spherical shapes with diameters of 10.2±0.7 nm.

FIG. 16A illustrates the 2D SAXS pattern and corresponding S(q) for Sys-IAₙ with n=15.

FIG. 16B illustrates the 2D SAXS pattern and corresponding S(q) for Sys-IAₙ with n=30.

FIG. 16C illustrates the 2D SAXS pattern and corresponding S(q) for Sys-IAₙ with n=50.

FIG. 16D illustrates the 2D SAXS pattern and corresponding S(q) for Sys-IAₙ with n=80.

FIG. 16E illustrates the 2D SAXS pattern and corresponding S(q) for the mixture of STV-IO and Au particles without Biotin-DNA.

FIG. 16F illustrates the 2D SAXS pattern for the mixture of STV-IO and Biotin-DNA without Au particles.

FIG. 17A illustrates S(q) as a function of temperature for Sys-IA₃₀.

FIG. 17B illustrates S(q) as a function of temperature for Sys-IA₃₀.

FIG. 18A illustrates the 2D SAXS pattern and corresponding S(q) for Sys-IA₃₀ with the mole ratio IO:Au:Biotin-DNA::1:1:7.

FIG. 18B illustrates the 2D SAXS pattern and corresponding S(q) for Sys-IA₃₀ with the mole ratio IO:Au:Biotin-DNA::1:1:60.

FIG. 18C illustrates the 2D SAXS pattern and corresponding S(q) for Sys-IA₃₀ with the mole ratio IO:Au:Biotin-DNA::1:5:75.

FIG. 18D illustrates the 2D SAXS pattern and corresponding S(q) for Sys-IA₃₀ with the mole ratio IO:Au:Biotin-DNA::5:1:75.

FIG. 19 is a schematic illustration of the assembly system for linker assisted hybridization of binary nanoparticles or nanoparticles and protein entities.

FIG. 20A illustrates the 2D SAXS pattern and corresponding S(q) for Sys-IALₙ, for n=0.

FIG. 20B illustrates the 2D SAXS pattern and corresponding S(q) for Sys-IALₙ, for n=30.

FIG. 20C illustrates the 2D SAXS pattern and corresponding S(q) for Sys-IALₙ, for n=70.

FIG. 20D illustrates the 2D SAXS pattern and corresponding S(q) for Sys-IALₙ, for n=170.

FIG. 21A illustrates the calculated 8(q) for CsCl using Powder Cell in a two-atom system with an atom number ratio (AR) of 2.6.

FIG. 21B illustrates the calculated S(q) for α-ReO₃ using Powder Cell in a two-atom system with an atom number ratio (AR) of 2.6.

FIG. 21C illustrates the calculated S(q) for AuCu₃ using Powder Cell in a two-atom system with an atom number ratio (AR) of 2.6.

FIG. 21D illustrates the calculated S(q) for La₂O₃ using Powder Cell in a two-atom system with an atom number ratio (AR) of 2.6.

FIG. 21E illustrates the calculated S(q) for NaTl using Powder Cell in a two-atom system with an atom number ratio (AR) of 2.6.

FIG. 21F illustrates the calculated S(q) for NaCl using Powder Cell in a two-atom system with an atom number ratio (AR) of 2.6.

FIG. 21G illustrates the calculated S(q) for ZnS using Powder Cell in a two-atom system with an atom number ratio (AR) of 2.6.

FIG. 21H illustrates the calculated S(q) for CaF₂ using Powder Cell in a two-atom system with an atom number ratio (AR) of 2.6.

FIG. 22A illustrates the magnetic field-dependent 2D SAXS pattern and corresponding S(q) for Sys-IA₃₀.

FIG. 22B illustrates the magnetic field-dependent 2D SAXS pattern and corresponding S(q) for Sys-IAL₁₃₀.

FIG. 23A illustrates the 2D SAXS pattern and corresponding S(q) for Sys-SAₙ, for n=15.

FIG. 23B illustrates the 2D SAXS pattern and corresponding S(q) for Sys-SAₙ, for n=18.

FIG. 23C illustrates the 2D SAXS pattern and corresponding S(q) for Sys-SAₙ, for n=30.

FIG. 23D illustrates the 2D SAXS pattern and corresponding S(q) for Sys-SAₙ, for n=50.

FIG. 24A illustrates the 2D SAXS pattern and corresponding S(q) for Sys-Q77ₙ, for n=3.

FIG. 24B illustrates the 2D SAXS pattern and corresponding S(q) for Sys-Q77ₙ, for n=15.

FIG. 24C illustrates the 2D SAXS pattern and corresponding S(q) for Sys-Q77ₙ, for n=30.

FIG. 24D illustrates the 2D SAXS pattern and corresponding S(q) for Sys-Q75ₙ, for n=3.

FIG. 24E illustrates the 2D SAXS pattern and corresponding S(q) for Sys-Q75ₙ, for n=30.

FIG. 24F illustrates the 2D SAXS pattern and corresponding S(q) for Sys-Q75ₙ, for n=50.

FIG. 25A depicts the photoluminescence of Sys-Q77ₙ, including the control system (a mixture of Q7 and Q7 without biotin-DNA), for the n=18, 30 and 50 systems.

FIG. 25B illustrates the enhancement factor (EF) of Sys-Q77 against the surface-to-surface distance between Q7 and Q7.

FIG. 25C illustrates the photoluminescence of Sys-Q75ₙ, including the control system (a mixture of Q7 and Q5 without biotin-DNA), for the n=18, 30 and 50 systems.

FIG. 25D illustrates the enhancement-to-quenching factor (EQF) of Sys-Q75 against the surface-to-surface distance between Q7 and Q5.

FIG. 26 illustrates the 2D SAXS pattern and corresponding S(q) for Sys-QPD₃₀ at different temperatures.

FIG. 27 illustrates the photoluminescence of Sys-QPD.

FIG. 28 is a schematic illustration of the three-step strategy for DNA functionalization of hydrophilic and hydrophobic nanoparticles (f—denotes the number of grafting DNA on the nanoparticles).

FIG. 29 are schematics and SEM images for biotinylated DNA-tethered palladium nano-cube (NC), octahedron (NO), and dodecahedron (ND) that were coated with PVP.

FIG. 30 is a schematic, TEM image (inset is HRTEM), and hysteresis loop for biotinylated DNA-grafted IO nanoparticles originally capped by oleic acid.

FIG. 31 is a schematic, TEM image (inset for if HRTEM), and photoluminescence spectra for biotinylated DNA-attached CdSe/ZnS QDs (QD525, denoted by Q5, and QD605, denoted by Q6) and CdTe/ZnS QDs (Q705, denoted by Q7). TEM image is for Q7.

FIG. 32 is a schematic, TEM (for 10 nm Au nanoparticles), and UV-Vis spectra for thiolated DN-functionalized Au nanoparticles, including 10, 15, 20 nm, originally capped by citrate.

FIG. 33A illustrates plots of shape-dependent structure factors (S(q)) extracted from SAXS patterns of direct hybridization (DH) systems with short DNA.

FIG. 33B illustrates in the top portion the Au nanoparticle size-dependent S(q) evolution of ND-Au DH systems, including PD hybridized with 15 nm and 20 nm Au.

FIG. 33C illustrates the effects of nanoparticle shape on the correlation length (ξ) of binary systems assembled by shaped and spherical NPs.

FIG. 33D is a plot showing the nearest neighbor particle surface-to-surface distance (Dₛₛ), as illustrated by inset, for ND-10 nm Au systems.

FIG. 34A illustrates plots of shape-dependent structure factors (S(q)) extracted from SAXS patterns of DH systems for Fe₂O₃ (denoted as FeO in figures) and Au nanoparticles. (1): S(q) for non-specific interaction induced Fe₂O₃ aggregates. (2): A DNA base number (N)-dependent evolution of S(q) from the single component Phase-F to a DNA-directed Au-IO binary superlattice upon introducing Au nanoparticles, tethering DNA direct complementary to that on IO surface, into Sys_FeO; (3) S(q) for a DH system assembled by STV and Au nanoparticles with longer and shorter DNA.

FIG. 34B is a 3D schematic illustration for structure switch between Phase_F and Phase_D via introducing Au nanoparticles or elevating temperature.

FIG. 34C shows the assembly kinetics for Phase-F and Phase-D. The inset is a 2D schematic for phase-D.

FIG. 34D is a plot of the Dₛₛ for IO-Au direct hybridization systems and the α calculated from geometrical consideration based on the Dₛₛ values as a function of N. Inset illustrates the definition of Dₛₛ and α in the Au-IO supperlattice.

FIG. 34E shows the experimental configuration for SXAS measurement in a magnetic field (top) and the S(q) magnetic response (bottom) of the IO-Au direct hybridization systems.

FIG. 35A is a plot showing component-dependent S(q) evolution of DH systems for QD-Au nanoparticles.

FIG. 35B shows the DNA-spacer length dependent S(q) evolution of Q7-Au systems (top) and S(q) of a well ordered Q7-Au system, which involves both flexible and rigid DNA regions (bottom).

FIG. 35C is a plot showing the change of compositional order parameter (η) and correlation length (ξ) with DNA base number (N) for DH Q7-Au systems. The inset sketches the compositional order-to-disorder transition with η from 1 to 0 in a CsCl lattice formed in the binary Au and QD systems.

FIG. 35D is a plot of Dss for QD-Au DH systems.

FIG. 35E is a plot of steady-state and time-resolved PL spectra collected from Q7-Au direct hybridization systems.

FIG. 35F illustrates a sketch of a CsCl lattice formed by Q7 and Q5 directed by DNA. FIG. 35F also shows a plot of the lifetime (τ) for donor (Q5) and acceptor (Q7) in the free-dispersed states and superlattice Q7_Q5₃_{_{—}}₃.

FIG. 36A is a phase diagram for the heterogeneous binary—10 nm nanoparticle systems.

FIG. 36B is a diagram showing an example (N=30, DH systems) for the predictable interparticle center-to-center distances (D_{cc}) for heterogeneous binary systems.

### DETAIL DESCRIPTION

In accordance with aspects of the present invention, applicants have developed a general strategy for multi-component DNA-guided three-dimensional (3D) assembly of functional nanoparticles. The disclosure demonstrates a generally applicable strategy of how to functionalize nanoparticles with DNA, independent of the composition of the material or the shape of the nanoparticles. The disclosure further demonstrates a programmable assembly of the DNA-functionalized nanoparticles into predefined multi-dimensional and multi-component, such as, but not limited to, magnetic (Fe₂O₃ and other magnetic materials), plasmonic (Au and other metals), photonic (quantum dot, QD), and catalytic (Pd, Pt, and others) materials, and protein (such as STV) structures. Described herein is a general strategy for DNA-mediated self-assembly of multicomponent functionalized nanoparticles into three-dimensional (3D) ordered superlattices. Also described are exemplary embodiments of DNA-mediated heterogeneous assemblies of nanoparticles including new phases of known nanoparticle assemblies.

### (A) DNA Functionalization

The generally applicable strategy either allows for removal of the high affinity ligands that bind to the nanoparticle surface and their replacement with other ligands that do allow for subsequent functionalization with biological groups (mostly for hydrophilic nanoparticles), or provision of an additional ligand layer that allows for further functionalization with biological groups (mostly for hydrophobic nanoparticles), which can prevent irreversible and uncontrolled aggregation of nanoparticles while preserving their unique structures and physical properties. Such nanomaterials can then be applied in various programmable assembly strategies.

The general strategy for multi-component DNA-guided 3D assembly of functional nanoparticles is described herein. First, a generally applicable strategy of how to functionalize nanoparticles with DNA, independent of the composition of the material or the shape of the nanoparticles, will be described. For DNA functionalization, there is provided a facile method for the synthesis of non-commercially available nanoparticles with uniform size and shape. In a second step, either the original high affinity ligands are replaced by or additional ligands are provided with carboxylic acid functional groups. In a third step, 1-ethyl-3-[3-dimethylaminopropyl]-carbodiimide hydrochloride (EDC)-assisted chemistry is adapted to covalently conjugate streptavidin onto the particle surface due to the reaction between the carboxylic (COOH) groups of the ligands and the primary amine (NH₂) groups that are abundant on the STV surface. Finally, biotinylated-DNA is coupled with STV on the particle surface due to the strong and specific affinity of biotin to STV. This functionalization strategy is very versatile and robust. Certain examples demonstrate how to assemble organized superstructures with iron oxide (IO; such as magnetic Fe₂O₃), plasmonic (Au), photonic (QD), and catalytic (Pd) materials, and protein (STV), as well as combinations thereof. Also demonstrated is that these ordered structures possess rich phases that until now could not be obtained using the current state of the art in nanomaterial assembly approaches.

The methods of the present disclosure provide various examples to illustrate the general process of the invention for nanoparticle synthesis and subsequently DNA functionalization. Depending on the capping agent used for their synthesis, the nanoparticles can be divided into two classes, namely, hydrophilic and hydrophobic. For hydrophilic nanoparticles, the initial step is to first replace the original ligand by mercapto acid (MA), e.g., mercaptoundecanoic acid, and thereafter to conjugate it with STV, and then finally couple it with biotinylated-DNA. For hydrophobic nanoparticles, the initial step is to either replace the original ligands or provide additional ligands. In one embodiment, the initial step is to treat the nanoparticles with one or more amphiphilic polymers, such as lipid-PEG carboxylic acid, followed hr a conjugation with STV and coupling with biotinylated-DNA. The general procedure is shown in FIG. 28.

To demonstrate the universal applicability of this strategy with respect to the hydrophilic nanoparticles, palladium nanoparticles with different shapes are used as examples Palladium nanoparticles are important for hydrogenation catalysis. To demonstrate the universal applicability of this strategy with respect to the hydrophobic nanoparticles, the iron oxide (IO) capped with oleic acid (OA) and quantum dots (QD) capped with trioctylphosphine (TOPO) nanoparticles are used as examples. Iron oxide is a typical magnetic material and QD can be used as highly efficient luminescent nanocrystals.

### (B) Assembly of 3D Ordered Structure by Multi-Component Functional Nanoparticles

Once nanoparticles are successfully encoded with DNA, it is possible to either hybridize DNA-encoded nanoparticles or nanoparticles and proteins, independent of the particle's component, size, or shape, into 3D aggregations due to the specific interaction of DNA. The 3D ordered phases can be obtained by carefully controlling the interplay of interparticle attraction and repulsion energies, which can be experimentally achieved in a variety of ways, such as by controlling DNA sequence length, number and structure of DNA molecules, and DNA structure hybridization temperature.

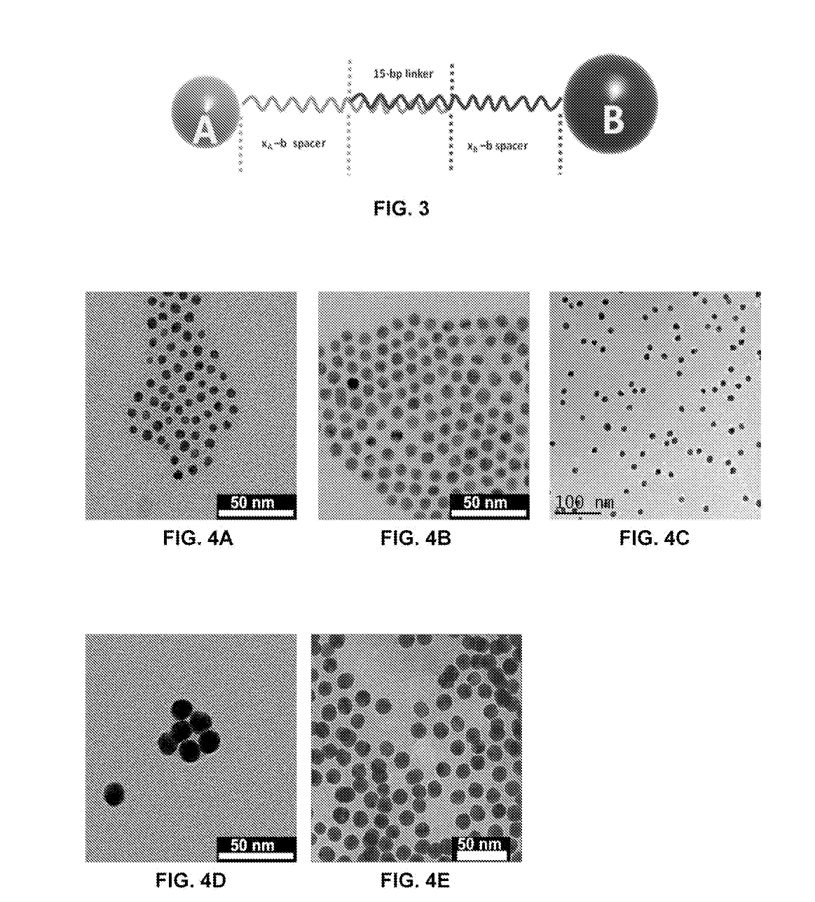

FIGS. 3 and 19 show a schematic illustration of an assembly system for direct hybridization (DH) and linker hybridization (LH) of binary nanoparticles, or nanoparticles and proteins, respectively. In a DH assembly system, nanoparticles can be functionalized with DNA that has two functional parts. One is non-complementary and forms the internal spacer part, which is designed to tune the repulsive interaction between particles, and the other is complementary, forming the outer recognition sequence part, and which provides the attraction interaction for nanoparticle assembly. The spacer part on particle A (B) can be designed as Xₐ (X_{b}) poly T bases and is denoted Xₐ−b (X_{b}−b) spacer in FIG. 3. The total base number (N) is defined as X_{A}+X_{B}−b. Alternatively, in a LH assembly system, nanoparticles can be functionalized with DNA that has two functional parts, but neither one complementary to provide the attraction interaction for nanoparticle assembly. However, while the outer spacer regions are non-complementary to each other, they are complementary to the respective base ends of a ssDNA linker, which has a central flexible part (base number denoted by Lₙ−b) separating the two ends. N is defined as X_{A}+X_{B}+Lₙ−b in LH systems. Generally, DH systems reveal quicker assembly kinetics in comparison with LH systems involving similar DNA length. While the LH strategy proves more flexible for system design, for example, regulation of the interparticle distance can be achieved by simple tuning linker base number without changing grafting DNA types.

The following examples are included to demonstrate preferred embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Synthesis of Palladium Nanoparticles with Controlled Shapes (Octahedral, Cubic, and Dodecahedral) and Size

Palladium (Pd) nanoparticles were synthesized in an aqueous solution by a modifying the procedure described in Lim et al. (2009). In the original reported procedure, only Pd nanoparticles with cubic shape were obtained. Here, two new shapes (octahedral and dodecahedral) were obtained by either changing the KBr concentration or by using potassium iodide (KI), which was an important modification of the reported procedure.

Water soluble inorganic Pd salts, such as Na₂PdCl₄ or K₂PdCl₄, were used as a palladium source. Poly-vinyl-pyrrolidone (PVP) (having a typical molecule weight (M.W.) ranging from ˜30,000 to 100,000) was used both as reluctant and surfactant. Alkali metal bromides or iodides, such as NaBr, KBr, NaI, and KI, were used as shape-controlling agents. Bromides were used for the synthesis of nano-octahedrons (NOs), nanocubes (NCs), and nanododecahedrons (NDs), while iodides were used for the synthesis of dodecahedrons. In a typical synthesis procedure, a mixture of Pd salt and alkali metal halide was first heated to about 80-100° C. with a standard reflux system and kept at that temperature for about 30 minutes. Then a pro-heated PVP solution was injected into the mixture solution. The reaction was allowed to continue for about 3-5 hours. For the synthesis of Pd NOs, the mole ratio between Pd salt, bromide, and PVP was approximately 1: (3-30):(3-8) for temperatures around 80-90° C. and approximately 1:(3-15):(3-8) for temperatures around 90-100° C. For the synthesis of Pd NCs, the mole ratio between Pd salt, bromide, and PVP was about 1:(15-30):(3-8) for temperatures around 90-100° C. For the synthesis of Pd NDs, if bromide was used in the reaction, the mole ratio between Pd salt, bromide, and PVP was about 1:(30-60):(3-8) for temperatures around 80-100° C. The Pd NDs may also be obtained by introducing trace iodide to the reaction.

The mole ratio of Pd salt to bromide, iodide, and PVP can be around 1:(3-60):(0.01-0.1):(3-8) and reaction temperatures can be around 80-100° C. For the above three synthesis reactions, Pd salt concentration typically ranges between about 10 mmol/l to about 30 mmol/l. After the reaction the nanoparticle products were collected by centrifugation, and then purified by washing once with acetone and subsequently three times with ethanol or water. The as-obtained nanoparticles can be well dispersed in ethanol or water. The Pd nanoparticles obtained by such methods are uniform in shape with no more than 15% unexpected shape, and also have a narrow size distribution (<10%). The yield of nanoparticle for NOs. NCs, and NDs are about 70%, 50%, and 40%, respectively, calculated from the transformation of Pd from salt form to nanoparticle form.

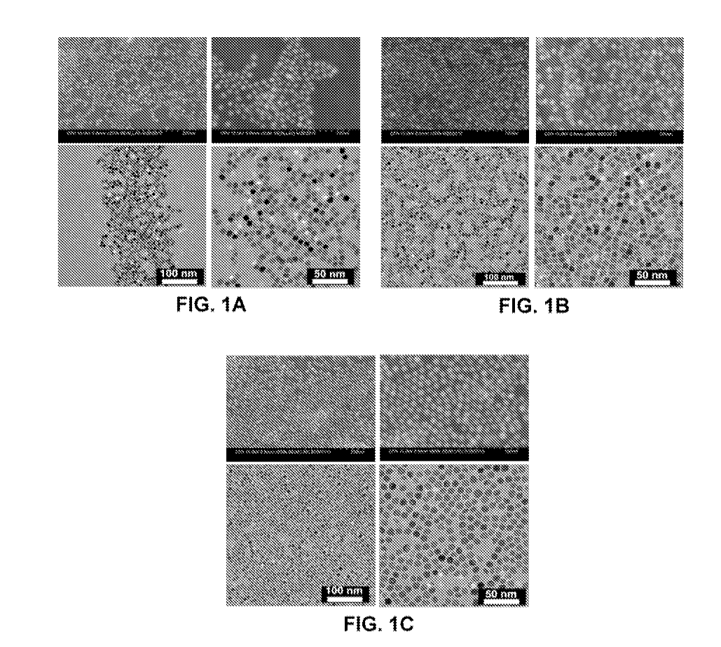

By regulating the synthesis parameters, such as reactant ratio and concentration, temperature, and reaction time, one can control the edge size of NOs, NCs, and NDs ranging from about 6 to 13 nm. Generally, higher temperature, lower halide concentration, and longer reaction duration will produce bigger nanoparticles. FIGS. 1A, 1B, and 1C show scanning electron microscopy (SEM) and transmission electron microscopy (TEM) images for the prepared Pd NOs, NCs, and NDs, respectively. The edge size of Pd NOs, NCs, and NDs are 8.6±0.8 nm, 10±1 nm, and 9±0.9 nm, respectively. The synthesis parameters for Pd nanoparticles shown in FIG. 1 are as follows: for NOs, [Na₃PdCl₄]=58 mM, mole ratio Na₂PdCl₄:KBr:PVP (M.W.˜50,000)=1:20:5, temperature=80° C., and the reaction time is about 3 h; for NCs, [Na₂PdCl₄]=58 mM, mole ratio Na₂PdClr:KBr:PVP (M.W.˜50,000)=1:20:5, temperature=100° C., and the reaction time is about 3 h; for NDs, [Na₂PdCl₈]=58 mM, mole ratio Na₂PdCl₈:KBr:PVP (M.W.˜50,000)=1:50:5, temperature=100° C., and the reaction time is about 3 h.

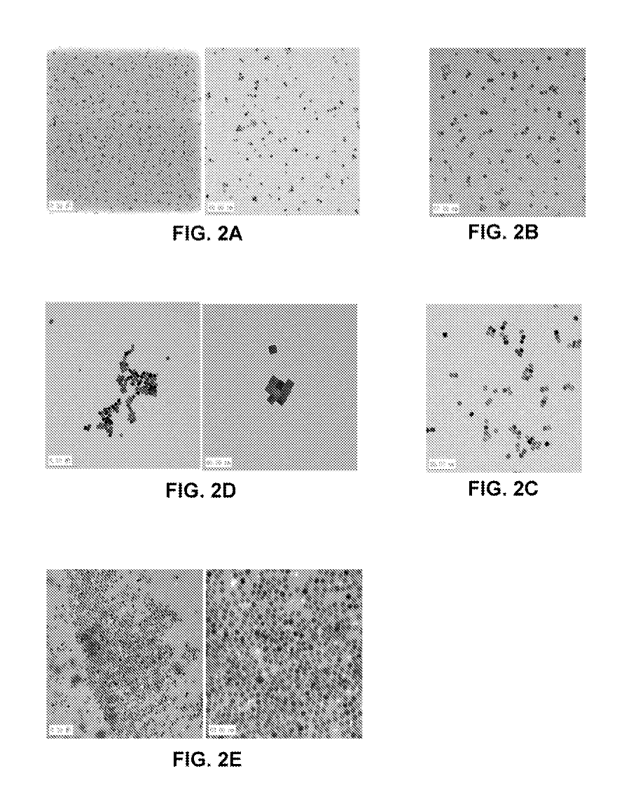

To grow larger palladium (Pd) nanoparticles, a seed-mediated method was developed wherein small sized nanoparticles are used as seeds and Pd (0) is reduced and deposited onto the surface of the seeds. Generally, using such a method one can predictably produce Pd nanoparticles with good control of shape as well as with precise size control, even at the am level. The nanoparticle shape mainly depends on the ratio of Pd salt to bromide or iodide, and such ratios for synthesis of larger Pd NOs, NCs, and NDs are roughly the same as that described above for the synthesis of the corresponding nanoparticles. The nanoparticle sizes depend on the ratio between seeds and Pd salt, and a higher ratio of Pd salt will produce bigger nanoparticles. For instance, one can use small sized NCs as seeds to grow big sized NOs, NCs, and NDs. FIG. 2A shows Pd NCs with an edge size of 6±0.5 mm, for which the synthesis parameters are set as [Na₂PdCl₄]=58 mM, mole ratio Na₂PdCl₄:KBr:PVP (M.W.˜50,000)=1:20:5, temperature=100° C. Using such NCs as seeds, bigger NCs with edge sizes of 10±0.8 nm (FIG. 2B), 12±0.9 nm (FIG. 2C), 23±2.6 nm (FIG. 2D), and bigger NOs with an edge size of 15±1.3 nm (FIG. 2E) can be obtained.

The synthesis parameters for Pd nanoparticles shown in FIGS. 2A through 2E are as follows: for NCs, the growth solution is that [Na₂PdCl₄]=1 mM, mole ratio Na₂PdCl₄:KBr:PVP (M.W.˜50,000)=1:20:5, temperature 100° C., the reaction time is 3 h, and the mole ratio between 6 nm NCs seeds and Na₂PdCl₄ is 10⁵ for 10 nm NCs, 2-10⁵ for 10 nm NCs, and 2-10⁶ for 23 nm NCs; for NOs, the growth solution is that [Na₂PdCl₄]=1 mM, mole ratio Na₂PdCl₄:KBr:PVP (M.W.˜50,000)=1:10:5, temperature 80° C., the reaction time is 3 h, and the mole ratio between 6 nm NCs seeds and Na₂PdCl₄ is 1.5*10⁵ for 15 nm NOs.

### Example 2

### Functionalization of Pd Nanoparticles with Mercapto Acid by a Ligand-Exchange Process

The PVP cap on the surface of Pd nanoparticles, including NOs, NCs, and NDs, can be replaced with mercapto acid by a ligand-exchange process. The carbon number of alkane can range between about 2 to 18, but a longer carbon chain length may be better for stabilizing the nanoparticles. The thiol group number in MA can be one, two, or more. The typical ligand-exchange process can be described in three steps. First, the pH value of the freshly prepared PVP-capped Pd nanoparticles in aqueous solution was adjusted to about 6-9 by buffer, which contains about 0.01% to 1% (by volume) surfactant. The buffer can be phosphate buffer, borate buffer, etc., and the pH value can range between about 6-9. The surfactant can be Tween (such as Tween 20), Triton (such as Triton 100), sodium dodecyl sulfate (SDS) and so on. Mercapto acid (MA) in ethanol, for instance 11-mercapto-undecanoic acid (MUA), is mixed with the above solution.

The mole ratio of mercapto acid can be about 10⁵ to 10⁷ times to that of nanoparticles depending on the surface area of nanoparticle, e.g., for Pd nanocubes with an edge size of 10 nm the ratio can be about 2*10⁵. In the second step, the above mixture was incubated at about 50-90° C. for about 3 to 12 hours after brief sonication for about 20 minutes to 1 hour. Finally, the as-functionalized nanoparticles were purified by a centrifugation-wash cycle procedure, where the particles are washed two times with ethanol and three times with the above buffer with surfactant. Such a functionalizaion procedure produces MA-capped Pd nanoparticles which are well dispersed in buffer or aqueous solution. This functionalization method is robust and can also be applied for hydrophilic materials other than Pd and other surfactants than PVP. The materials can be gold, silver, platinum, and so on. The original surfactant can be very broad and their charge can be varied from negative charge, such as citrate, positive charge, such as cetyltrimethylammonium bromide (CTAB) cetylpyridinium chloride (CPC), poly-diallyl-dimethylammonium chloride (PDDA), to neutral charge, such as Pluronic P-123, Carboxymethyl Cellulose Sodium (CMC).

### Example 3

### Conjugation of Pd Nanoparticles with STV

The as-prepared MA-capped Pd nanoparticles (or other component nanoparticles) can be conjugated with STV by formation of an amide bond between carboxylic groups on the nanoparticles, provided by the ligand, and primary amine groups of STV through 1-ethyl-3-[3-dimethylaminopropyl]-carbodiimide hydrochloride (E-D) assisted chemistry. Typically. MA-capped Pd nanoparticles in buffer with pH about 6-8 are first mixed with freshly prepared EDC (about 0.1 mg/ml to 1 mg/ml), N-hydroxysulfosuccinimide (NHS, about 0.1 mg/ml to 1 mg/ml) and STV. The quantity of STV can be about 10 to 100 times that of the Pd nanoparticles. The mixture is allowed to incubate either at room temperature for about 1 to 4 hours or at 4° C. for about 6 to 12 hours. Finally, the nanoparticles are collected by a centrifugation-wash cycle procedure, when the particles can be washed three times by water or the above motioned surfactant-containing buffer. After purification, the nanoparticles are dispersed in surfactant-containing buffer.

### Example 4

### Functionalization of Pd Nanoparticles with Biotinylated-DNA

The as-prepared STV-capped Pd nanoparticles (or other component nanoparticles) were coupled with biotinylated-DNA because of the strong and specific affinity of biotin to STV. The DNA sequence from 5′ to 3′ of the recognition part on A has a sequence TAC TTC CAA TCC AAT [SEQ 1] and is complementary to the sequence on B, which is ATT GGA TTG GAA GTA [SEQ 2] from 5′ to 3′. The system was denominated as Sys-A_{Da/Ea}XaB_{Db/Eb}Xb, where the subscript Dₐ and D_{b} or Eₐ and E_{b} denote the diameter or emission wavelength (for QD) of particle A and B, respectively.

The STV-capped nanoparticles were mixed with biotinylated-DNA, which amounts can be used to control DNA number on the particle surface, and the mixture was allowed to incubate for several hours at room temperature. Finally, the nanoparticles were collected by a centrifugation-wash cycle procedure, where the particles were washed three times by water or the above-mentioned surfactant-containing buffer. After purification, the nanoparticles were dispersed in surfactant-containing buffer.

The three kinds of Pd nanoparticles had uniform shape and size and displayed the similar volume corresponding to about 11 nm spherical particles as illustrated in FIG. 29. The attached DNA number (f) was typically 15-25.

### Example 5

### Synthesis of IO and QD Particles

The synthesis of iron oxide (IO; e.g., Fe₂O₃) nanoparticles with sizes from about 4 to about 16 nm followed procedures published by Hyeon, T., et al. (Journal of the American Chemical Society, 2001. 123(51): p. 12798-12801; incorporated herein by reference). Synthesis of quantum dots (QDs) with emission wavelengths of 400 nm to 780 nm followed procedures published in Dabbousi (1997) and Medintz. I. L., et al., (Nature Materials, 2005. 4(6): p. 435-446; incorporated herein by reference).

### Example 6

### Functionalization of IO and QD with Carboxylic Acid Groups

For the first method, iron oxide (Fe₂O₃) nanoparticles or quantum dots (QDs) dispersed in an organic solvent, such as toluene or chloroform, were first mixed with MA (usually 3-mercaptopropionic acid (MPA)) in ethanol or methanol solvent. Then the mixture was heated at about 5° C. to 70° C. for about 4 to 12 hours after brief sonication for about 5 to 30 minutes. Finally, the nanoparticles were collected by a centrifugation-wash cycle procedure, where the particles can be washed three times by water or the above mentioned surfactant-containing buffer. After purification, the nanoparticles were dispersed in surfactant-containing buffer. This is similar to the procedure for QD published by Kang, S. H., at al., (Applied Physics Letters, 2008. 93(19): p. 191116-1 to -3, which is incorporated herein by reference in its entirety).

For the second method, Fe₂O₃ or QD dispersed in an organic solvent, such as toluene or chloroform, were first mixed with amphiphilic polymers, such as poly(maleic anhydride alt-1-tetradecene), lipid-PEG carboxylic acid, which have hydrophobic chains interacting with ligands on the nanoparticles and carboxylic acid groups for further functionalization. Then the mixture was incubated for about 2 to 4 hours at room temperature. After complete evaporation of the organic solvent, the residual solid was purified by a centrifugation-wash cycle procedure, where the particles are washed three times by water or buffer with pH about 7 to 9, such as borate, TBE. After purification, the nanoparticles were dispersed in water or buffer. A similar procedure has been reported by Pellegrino. T., et al. (Nano Letters, 2004. 4(4): p. 703-707; incorporated herein by reference in its entirety).

### Example 7

### Conjugation with STV and biotinylated-DNA

These two steps are nearly the same as those described above for Pd nanoparticles. Although STV has been used to functionalize Fe₂O₃ (Lee 2006 and Shen, T. T, et al., Bioconjugate Chemistry, 1996. 7(3): p. 311-316, which is incorporated herein by reference in its entirety) and QD (Glazer, A. N., Bioconjugate technique-Hermanson, GT. Nature, 1996. 381(6580): p. 290-290, which is incorporated herein by reference in its entirety), the resultant nanoparticles have not undergone 3D crystallization.

Using the above procedure, uniform ˜10 nm (diameter) spherical nanoparticles of Fe₂O₃ capped with oleic acid (OA) having superparamagnetic properties were synthesized as illustrated in FIG. 30. Hydrophobic commercially-available QD capped with trioctylphosphine oxide (TOPO) of three different emission peaks (λm) centered at 525 (core-shell CdSe/ZnS), 605 (core-shell CdSe/ZnS), and 705 nm (core-shell CdTe/ZnS) were also synthesized using the above procedure as illustrated in FIG. 31. All the particles showed a slightly elongated shape and the hard-core particle size was about 2˜3, 4˜6, and 6-7 nm. Citrate-capped Au nanoparticles of three different size (−10, 15, 20 nm) were also synthesized with dense thiol-DNA as shown in FIG. 32. The attached DNA number (f) on 16.8-nm Au, QD, and 10-nm IO nanoparticles is about 20 (45-60 for 10 nm Au), about 20-40, and 3-8, respectively.

### Example 8

### Particle Assembly

For particle assembly, a defined ratio of particles A and B was mixed in 10 mM phosphate buffer with 0.14 M NaCl, pH=7.1 at room temperature. The particles were allowed to assemble into aggregates for from several minutes to days, depending on the particle concentration. Subsequently the precipitates were split into two parts, one for melting temperature measurements and the other, after transferring into a capillary, for structure measurement. The melting temperature was determined using UV-Vis spectroscopy, monitoring the change in absorbance at the nanoparticles' predominant absorption peak. The structure of the assembly was analyzed by synchrotron-based small-angle X-ray scattering (SAXS), which was performed at the National Synchrotron Light Source X-9A beam line. If not specifically mentioned, the samples in the capillary were annealed at a temperature several (about 1 to 5) degrees below their melting temperature for ten minutes to several hours and then slowly cooled down to room temperature for several hours before SAXS measurements.

For SAXS data analysis, the scattering data were collected with a MAR CCD area detector and converted to 1D scattering intensity vs. wave vector transfer, q=(4π/λ) sin(θ/2), where λ and θ are the wavelength of incident X-ray and the scattering angle, respectively. The structure factor S(q) was calculated as Iₐ(q)/Iₚ(q), where Iₐ(q) and Iₚ(q) are background corrected 1D scattering intensities extracted by angular averaging of CCD images for assembled systems and un-aggregated particles, respectively. The peak positions in S(q) are determined by fitting to the Lorentzian equation.

To analyze the structure of the assembly, the peak position ratio (Qx/Q1) from the structure factor as well as the relative peak intensity are initially used to propose possible structure models, and then such proposed models are compared with first peak positions (q1) to calculate the nearest neighbor particle center-to-center distances (D_{cc}M) in the assembly, and finally the most probable model is obtained by comparing the D_{cc}M and the distances (D_{cc}C) calculated in real space from the designed system configuration.

### Example 9

### Au and Au with Different Sizes

Thiolated DNA-capped Au nanoparticles (TA) (functionalization methods can be found in the reports of Nykypanchuk 2008 and Park 2008) and biotinylated DNA-capped Au nanoparticles (SA) (the functionalization method is similar to that of Pd nanoparticles) are used as particle models to illustrate the phase behavior for the hybrid system composed of Au nanoparticles with different sizes and surface chemistries. FIGS. 4A, 4B, 4C, and 4D show TEM images for the four kinds of TA with corresponding diameters of 6.2±1 nm, 8.8±1.7 nm. 12.5±1.8 nm, and 14.7±2 nm, each of which was used to hybridize with DNA-biotin-STV capped Au (SA) with diameter of 16.6±1.5 nm (FIG. 4E). For the hybrid system 9-nm TAu with 16.8-nm SAu, four kinds of internal spacer sets (Xa-Xb), namely, 15-3, 15-15, 35-35, and 65-65, were used, and the systems were denominated as Sys-AA9ₙ, and n=18, 30, 50, and 80, respectively. For the hybrid systems 6-nm, 12.5-nm, and 15-nm TA with 16.8 nm SA, all the internal spacer sets (Xa-Xb) were designated as 35-35, and the systems were accordingly nominated as Sys-AA6₅₀, Sys-AA12₅₀, and Sys-AA15₅₀.

FIGS. 5A, 5B, 5C, 5D, 5E, 5F, and 5G show the 2D SAXS pattern and corresponding S(q) of the Sys-AA9₁₈, Sys-AA9₃₀, Sys-AA9₅₀, Sys-AA9₈₀, Sys-AA6₅₀, Sys-AA12₅₀, and Sys-AA15₅₀, respectively. Here, S(q)=Iₐ(q)/Iₚ(q); the melting system was used for Iₚ(q). Interestingly, the first peak in Sys-AA9 and Sys-AA6, which contain particles of big size difference, has weaker intensity compared with the second one; while for Sys-AA12 and Sys-AA15 the first peaks are always the strongest ones, which is the same as the reported results of single component systems (Nykypanchuk 2008 and Park 2008). By fitting the 1D scattering curves from the melting system, the size distribution of the particles in the system can be obtained.

FIG. 5H shows the 2D SAXS pattern of the melting Sys-AA9₅₀, and the gray and black 1D curves correspond to the integrated scattering intensity of melting and assembled Sys-AA9₅₀, respectively. The fitting of the melting curve (FIG. 5I) using the Irena 2 macros package gives two particle size distributions with diameters of about 9 nm and about 16 nm, which confirms that the system was assembled by two different sizes of nanoparticles. FIG. 5J shows the 2D SAXS pattern of the melting Sys-AA15₅₀, and the fitting of the melting curve, which indicates the single size distribution due to the similar size of the two particles in this system.

To analyze the assembled structure, first consider Sys-AA12 and Sys-AA15, where Qₓ/Q₁=1:√3:√7 and such ratios correspond to a body-centered cubic (BCC) structure. The BCC structure is expected for the hybrid system with two types of Au nanoparticles of similar size, and the result is in coincidence with the reports of Nykypanchuk 2008 and Park 2008. For Sys-AA9ₙ and Sys-AA6₅₀, all the systems have similar structures and Sys-AA9₅₀, was used to analyze their structure. In Sys-AA9₅₀, Qₓ/Q₁=1:1.71:3.0:4.1:4.95:6.0; interestingly, Qₓ/Q₂=1:1.75:2.19:2.89:3.5. Considering the two ratios, the structure is similar to a type of face-centered cubic (FCC) with Qₓ/Q₁ as 1:1.63:2.31:2.83:3.41 from diffraction planes (111), (220), (400), (422), (531), while the first extinction peak of (100) also appears.

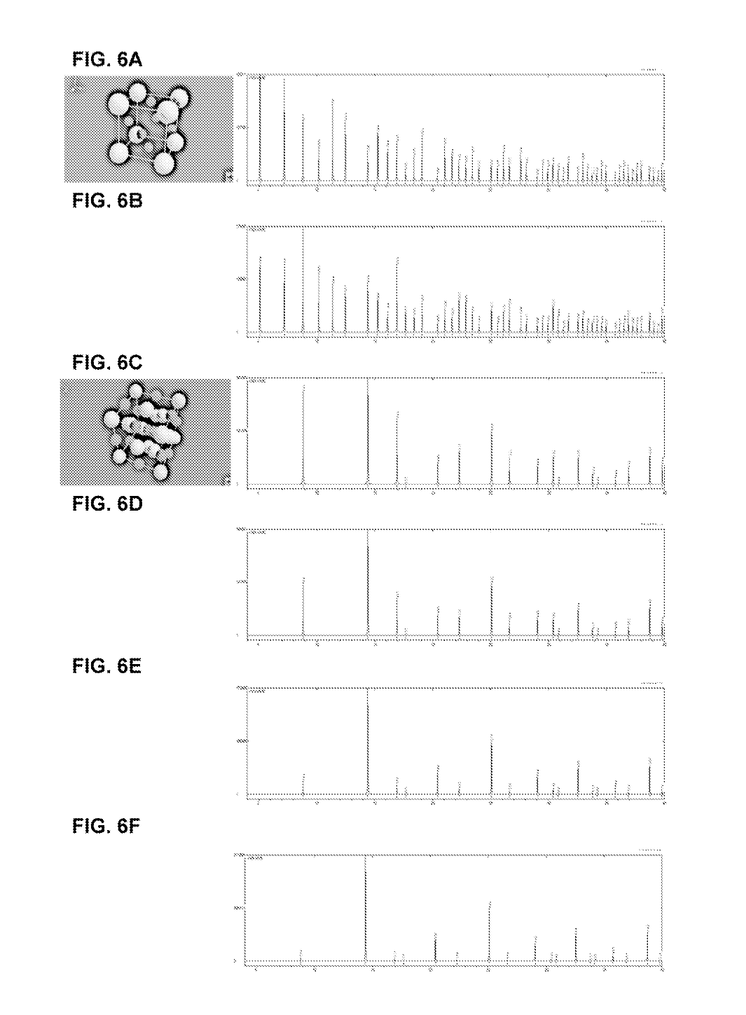

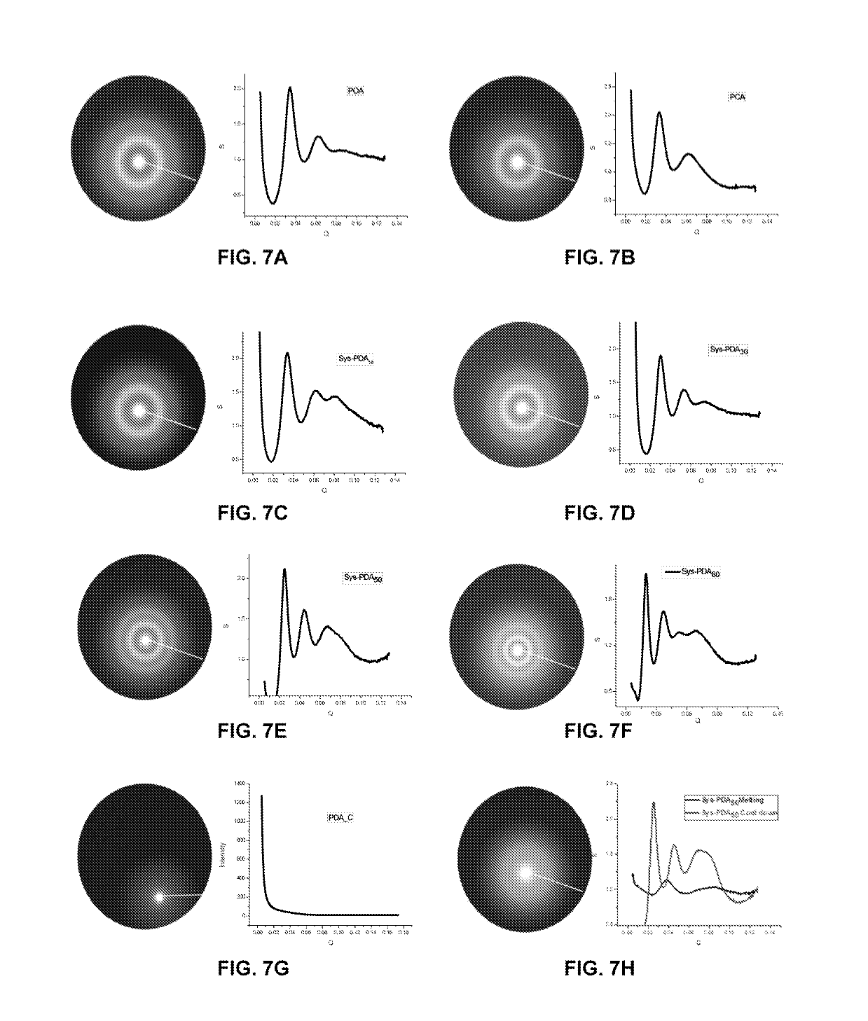

Two possible structure models, similar to the crystalline organizations of either Cu₃Au or NaTl, are proposed for the system. The Cu₃Au phase corresponds to the Pm 3̅m space group with group number 221, and Cu sits in 3c sites, and Au sits in the 1a site. (See the schematic in FIG. 6A.) The NaTl phase corresponds to the Fd 3̅m space group with group number 227, and Na sits in 8a sites, and Tl sits in 8b sites. (See the schematic in FIG. 6C.) The scattering ability (Is) of the Au particles used is then calculated. The Is can be roughly estimated as

Is_{Au}_{_{—}}_{Ra}/Is_{Au}_{_{—}}_{Rb}=[(σ_{eAu}−σ_{ebuffer})*V_{Au}_{_{—}}_{Ra}]²/[(σ_{eAu}−σ_{ebuffer})*V_{Au}_{_{—}}_{Rb}]²

and σₑ=ρ*Z/M_{w}, where σₑ is the electron density of the particles, ρ is the material density, Z is the material atomic or molecular electrons, and M_{w} is the material atomic or molecular weight. The Is_{Au-16}/Is_{Au-6} and Is_{Au-18}/Is_{Au-9} were calculated as 295 and 31, respectively.

We then use an atomic system to calculate the S(q) using software PowderCell, where Cu₃Au and NaTl structures containing two atoms with an atom number ratio of 17 (˜√295, resembling Sys-AA6) and 5 (˜√31, resembling Sys-AA9) were used. The same lattice constant was used to calculate the S(q). The calculated S(q) for Cu₃Au structure are shown in FIGS. 6A and 6B, which correspond to atom number ratios (AR) of 17 and 5 (heavy atom at 1a site), respectively. FIGS. 6C and 6D accordingly correspond the calculated S(q) for NaTl structure with AR of 17 and 5 (heavy atom at 8a site). NaTl fits well with the experimental results. When the particles on the Na and Tl sites are the same, the NaTl structure degenerates to a BCC structure. FIGS. 6E and 6F show the calculated S(q) with AR of 2 and 1.5, corresponding to Sys-AA12 and Sys-AA15, respectively. The calculated results show that S(q) changes into a BCC structure with the decrease of the particle size difference. Therefore, the results suggest that all these systems, no matter the size difference, are actually in a NaTl structure. This structure has very recently been reported for the assembly system of Au nanoparticles and protein particles (Qβ phage capsid), where the two particles have the same size (Cigler, P. et al. Nature Materials, 2010. 9(11): p. 918-922, which is incorporated herein by reference in its entirety).

With the proposed NaTl structure, D_{cc}M in the assembly can be calculated using Q₁. For Sys-AA9₁₈, Sys-AA9₃₀, Sys-AA9₅₀, Sys-AA9₈₀, Sys-AA6₅₀, Sys-AA12, and Sys-AA15, Q₁ are correspondingly 0.0177, 0.0168, 0.0144, 0.0114, 0.0141, 0.0216, and 0.0204 Å⁻¹. For Sys-AA12 and Sys-AA15, D_{cc}M=√6*π/Q₁ since the first peak comes from (220) in NaTl structure, and the values are 35.6 and 37.7 nm, respectively. For Sys-AA6 and Sys-AA9. D_{cc}M=1.5*π/Q₁ since the first peak comes from (111) in NaTl structure, and the values are 26.6, 28.1, 32.7, 41.3, and 33.4 nm for Sys-AA9₁₈, Sys-AA9₃₀, Sys-AA9₅₀, Sys-AA9₈₀, and Sys-AA6₅₀, respectively.

To validate the proposed model, D_{cc} was estimated using the following methods. For the configuration shown in FIG. 3,

D_{cc}C=R_{A}+T_{xA-b}+T_{xB-b}+R_{B}−Δ(X_{A},X_{B},X_{bp},N_{D}),

where R_{A} and R_{B} correspond to the radius of particles A and B, T_{xA-b} and T_{xB-b} correspond to the characteristic length of X_{A}- and X_{B}-base ssDNA tethered on particles, and Δ is the DNA shrinkage length due to hybridization (roughly related to the X tethered base), X_{bp} is the hybridized base, and N_{D} the DNA coverage on the panicles. Here. R_{A}=4.5 nm and R_{B}=12.9 nm (considering STV has a diameter of 4.5 nm). Then T was estimated by the Daoud-Cotton blob model and the parameters used are: persistent length (Iₚ) as 1 nm; salt concentration (Cₐ) as 0.14 M; and the DNA number (N_{D}) on 6, 9, 12, 15, and 16.8 nm Au are 30, 65, 70, 100, and 20, respectively. A for different X_{A}-X_{B} sets was obtained from a known BCC structure assembled by all 9-nm Au nanoparticles, and we obtained Δ=3.8, 6.9, and 7.3 for 15-15, 35-35, and 65-65 X_{A}-X_{B} sets, respectively. Using the above model, the calculated D_{cc}C=25.8, 27.4, 31.1, 39.6, 30.6, 35.1, and 36.8 for Sys-AA9₁₈, Sys-AA9₃₀, Sys-AA9₅₀, Sys-AA9₈₀, Sys-AA6₅₀. Sys-AA12 and Sys-AA15, respectively. The D_{cc}C is consistent with D_{cc}M, which confirms the proposed NaTl structure.

These results indicate that all the Au—Au systems where at least one of the two particles is coated with STV, no matter the size difference, actually do have a NaTl structure. This is the first time that this kind of structure has been reported for bioassembled inorganic materials. This finding is unexpected, as previous assemblies of Au particles with thiolated DNA were always reported to form a CsCl structure (Nykypanchuk 2008 and Park 2008), which however, cannot be distinguished from the NaTl structure when using Au particles with same sizes for the assembly.

### Example 10

### Au and Catalytic Pd

Thiolated DNA-capped Au nanoparticles and biotinylated DNA-capped Pd nanoparticles of different shapes were used as particle models to illustrate phase behavior for the hybrid system of Au and Pd nanoparticles. Each type of Pd nanoparticle, including NOs, NCs, and NDs shown in FIGS. 1A through 1C, was used to hybridize with 9-nm Au nanoparticles to form Sys-POA, Sys-PCA, and Sys-PDA, respectively. For both Sys-POA and Sys-PCA, the X_{A}-X_{B} sets were designed as 3-15. For Sys-PDA, the X_{A}-X_{B} sets were designed as 3-15, 15-15, 35-35, and 65-65, and the systems were nominated as Sys-PDAₙ, with n=18, 30, 50, and 80, respectively.

FIGS. 7A through 7F show the 2D SAXS pattern and corresponding S(q) of Sys-POA, Sys-PCA, Sys-PDA₁₈, Sys-PDA₃₀, Sys-PDA₅₀, and Sys-PDA₈₀, respectively. Here, S(q)=Iₐ(q)/Iₚ(q), Iₚ(q) was obtained from the control system, which is the mixture of STV-capped Pd nanoparticles and thiol-DNA-capped Au nanoparticles without biotinylated-DNA. FIG. 7G gives the 2D SAXS pattern and corresponding Iₚ(q) for an example control system, Pd NDs and Au (PDA-C). The control system does not show any diffraction patterns, which indicates that the STV-Pd nanoparticles are stable and verify the DNA-mediation role for the assembly as well. Such Pd—Au systems show different phase behaviors with temperature from the reported Au—Au systems and the above-described Au—Au with different size systems, where the assembly becomes dissociated at the melting temperature (M_{T}) and show no diffraction peaks for SAXS. However, the Pd—Au systems still show several peaks even at tens of degrees higher than M_{T} determined by UV measurements. For example, FIG. 7H gives the 2D SAXS pattern and corresponding S(q) (black curve) for Sys-PDA₅₀ at 71° C., which shows two peaks centered at 0.0387 and 0.0795 Å⁻¹. Interestingly, when the system was cooled down, these two peaks would disappear and S(q) be restored back to the original state, as evidenced by the gray curve in FIG. 711.

The structures of the Pd—Au system can be determined by using similar structure analysis methods as described for the Au—Au system. All the Pd—Au systems could have similar structures due to their similar structure factors as shown in FIGS. 7A through 7F. Taking Sys-PDA₈₀ for example. Qₓ/Q₁=1:1.8:2.64:3.49, and such values resemble the peak position ratios, which are 1:1.73:2.65:3.46, of diffraction planes (110), (211), (321), and (422) to (110) of a BCC structure. The Is of the Pd and Au particles is then calculated.

Is_{Au}/Is_{Pd}=[(σ_{eAu}−σ_{ebuffer})*V_{Au}]²[(σ_{ePd}−σ_{ebuffer})*V_{Pd}]²,

and the Is_{Au}/Is_{Pd} was calculated as 0.63. According to their similar scattering ability, the BCC structure can either be CsCl or NaTl, but these two structures are impossible to distinguish as stated for Sys-AA.

Using the proposed CsCl or NaTl structure, D_{cc}M for the Pd—Au system are calculated using Q₁. For systems shown in FIGS. 7A through 7F, the Q₁'s are correspondingly 0.0340, 0.0330, 0.0330, 0.0303, 0.024, and 0.0198 Å⁻¹, and using D_{cc}M=√6*π/Q₁, the D_{cc}M are correspondingly 22.6, 23.3, 23.3, 25.4, 30.9, and 38.9 nm. For the calculation of D_{cc}C, the following parameters were used: R_{A}=4.5 nm for Au nanoparticle and R_{B}=10, 11, 11 nm (including STV) for NO, NC, ND, respectively; DNA number on Pd nanoparticle=20; and the other parameters are the same as that used for Au—Au system. For the above systems, the calculated D_{cc}C=23.2, 24.0, 24.0, 25.8, 29.6, and 38.3 nm, which agrees with the corresponding D_{cc}M. Not wishing to be bound by the theory, it appears that although CsCl and NaTl structures can't be distinguished in the present systems a NaTl structure is more reasonable for such STV-capped Pd and thiol-Au system considering the NaTl structure for STV-capped Au and thiol-Au system.

For the melted Sys-PDA₅₀ system (FIG. 7H), D_{cc}M is 16.2 nm using D_{cc}M=2*π/Q₁, since this system resembles an amorphous structure. This value approaches that of the distance (18.5 nm) between two DNA-capped PD particles. Therefore, the structure of the melted system actually comes from the aggregation of PD particles. Not wishing to be bound by the theory, it may happen that with the dissociation of the hybrid DNA at the melting temperature the Au particles release from the assembly due to the high repulsive interaction related to high DNA coverage, while the Pd particles in the assembly collapse together possible reasons including depletion interaction from DNA-Au particle, weak magnetic interaction from Pd particles themselves, weak repulsive interaction related to low DNA coverage, or high local concentration of Pd particles. However, this kind of Pd particle aggregation is reversible and the Pd particles can re-hybridize with Au particles to form the CsCl or NaTl structure.

The CsCl (or NaTl) structure is expected for the system assembled from two types of spherical nanoparticle with similar size, as evident by reports of Nykypanchuk 2008 and Park 2008 and the above-described Au—Au system. However, herein, three kinds of Pd polyhedrons are used to hybridize with spherical Au, so other structures than BCC, such as simple cubic (SC) for NC-Au and FCC for ND-Au, are expected due to the anisotropic shape effect. The only observed BCC phase possibly resulted from the actual loss of the anisotropic property of Pd polyhedrons because of their thicker capping soft molecular layers (typically 7 to 16 nm) in comparison with their hard core size (typically 4 to 6 nm). Such Pd—Au systems may find important applications in the catalysis area because of the good catalytic properties of Pd nanoparticles, unique plasmonic-related properties of Au nanoparticles and the quite open framework of the assembled structure.

FIG. 33A (top) shows the structure factor S(q) (symbols) extracted from SAXS patterns for three DH systems, accordingly corresponding to 10 nm Au hybridized with NC for NC Au₃_{_{—}}₁₅, with NO for NO Au₃_{_{—}}₁₅, and with ND for ND Au₃_{_{—}}₁₅. The three systems show similar structures, including similar first peak positions (q₁) accordingly centered at 0.0339, 0.0333, and 0.0345 Å⁻¹: however, their correlation length (ξ) depends on the particle shape and increases for shape being more spherical-like, as shown in FIG. 33C. For example, S increases from 37 nm to 46 and 52 nm when Pd nanoparticles change from NC to NO and ND. To verify the universality of this shape effect, a spherical Au—Au system was built, Au_Au₁₅_{_{—}}₁₅ is with similar DNA design. The S(q) is given in FIG. 33A (bottom), which indeed shows a larger ξ of 60 nm as well as a similar structure as Pd—Au systems. The nature of the driving force for the Pd—Au systems was examined A control system was created by a mixture of Au and Pd nanoparticles but lacking the recognition of DNA sequences. No aggregates formed in this system, which indicates that the STV-Pd nanoparticles are stable and also verifies the DNA-mediation role for the assembly.

DNA flexibility is necessary for the crystallization of DNA-Au Nanoparticles. The design of DNA with a certain length was found to really facilitate the ordering of shaped Pd—Au and spherical Au—Au, although the spherical systems can attain more profound ordered states than shaped systems. Take dodecahedron Pd and Au system for example with N from 45 to 145 in direct hybridization and N from 60 to 130 in linker hybridization systems. It was found that q₁ shifted to small values with increasing the N indicating the increase of the interparticle distances. At the same time, ξ increased from 56 nm to 124 nm and then decreased to 91 nm with N increased from 45 to 130 (ND_Au_{1.70}, with S(q) displayed in the middle panel of FIG. 338) and 145. In contrast, the spherical Au—Au reach a well ordered state for N as 90 with ξ of 310 nm, and this system is denoted by Au_Au_{1.30}, which S(q) is displayed in the bottom panel of FIG. 33B.

Using the CsCl lattice, the experimental S(q) can be fitted well, especially for Au_Au_{1.30}, as shown by black solid lines in FIGS. 33A and 33B. Due to the similar form factors (ΔP(q)) for 10 nm Au and 11 nm Pd nanoparticles, such binary CsCl lattices actually show BCC patterns with first peak from (110), which is the same as single component Au systems. One way to confirm this type of lattice is to adjust the ΔP(q), and thus a SC pattern with (100) as first peak will display. Other systems comprising components with distinct form factors were also constructed. Au size was increased from 10 nm to 15 nm and 20 nm while keeping ND nanoparticle size unchanged. FIG. 338 (top) shows S(q) for two representative systems for ND with 15 nm Au nanoparticles and ND with 20 nm Au nanoparticles. In comparison with systems for ND and 10 nm Au, a weak peak with q centered at 1/\sqrt{2}of the original first peak gradually emerges with increase Au nanoparticle size. This peak was assigned as (100) peak from a SC structure. Therefore, above all, the Pd and Au Nanoparticles formed a CsCl superlattice, as schematically shown in FIG. 33C (insert). Distinct from the expected NaCl or FCC-like phase, the only observed CsCl lattice could be resulted from the effective shape transformation from anisotropic to isotropic shape due to the thick capping soft molecular layers. The spherical-like particle favors such shape transformation, and thus favors the CsCl lattices because they are the stable structures for spherical binary DNA nanoparticles.

Based on the CsCl structure, the nearest neighbor particle surface-to-surface distance (Dₛₛ) for ND-Au systems was calculated. FIG. 33D summarizes the Dₛₛ for the ND and 10 nm Au systems, which displays a range from ˜12 to 30 nm. While the interparticle distances can be regulated by the change of ionic strength, it can also be achieved by varying the DNA length. The established DNA structure model allows us to predict the Dₛₛ. A Daoud-Cotton (DC) blob model and a worm-like chain (WLC) model were used to calculate the tethered DNA thickness and linker length, respectively. FIG. 33D shows that the model distances agree well with the experimental data, especially for systems with shorter length DNA, and the accuracy is limited in ˜12%. Such Pd—Au systems could be attractive for optical and catalytic-related studies due to the intrinsic merits of Pd and Au nanoparticles, possible energy transfer between them, and the quite open framework of the assembled structure.

### Example 11

### Au and Fluorescent QD

First take 9.0-nm thiolated DNA-capped Au nanoparticles and biotinylated DNA-capped QD with an emission wavelength centered at 705 nm (denoted Q7) as an example to illustrate the phase behavior for the hybrid system of Au and QD nanoparticles. The systems were obtained by mixing DNA-Au with biotin-DNA first and then with STV-QD, and the mole ratio of QD to Au and biotin-DNA is 1:1:40. The size and shape of the QD was characterized by TEM and SAXS. FIG. 8A shows the TEM images of Q7, where the QD has an elongated shape. The size distribution histogram of Q7 gives the long axis length and short axis length as 14±2.5 nm and 6±1.5 nm, respectively. FIG. 8B shows the Iₚ(q), the fitting, and size distribution for Q7. The fitting gives two size distributions, 13±1.5 nm and 6.1±1.1 nm, which accordingly corresponds to the long and short axis of Q7. For this hybrid system, the X_{A}-X_{B} sets were designated as 0-15, 3-15, 15-15, 35-35, and 65-65, and the systems were denominated as Sys-Q7Aₙ, with n=15, 18, 30, 50, and 80, respectively.

FIGS. 9A through 9E give the 2D SAXS pattern and corresponding S(q) for Sys-Q7Aₙ, and the images 9A through 9E corresponds to n=15, 18, 30, 50, and 80, respectively. Here, S(q)=Iₐ(q)/Iₚ(q), Iₚ(q) was obtained from either the control system or the melting system. The S(q) shows that the first peak intensity is weaker than the second one for all the system, and becomes much weaker for longer DNA spacer. The control system is the mixture of STV-capped QD and Thiol-DNA capped Au nanoparticles without biotinylated-DNA. FIG. 9F gives the 2D SAXS pattern and its corresponding Iₚ(q) for a control system of Q7 and Au (Q7A-C), which does not show any diffraction patterns.

To investigate the temperature-dependent phase behavior. Sys-Q7A₃₀ without pre-annealing was selected as an instance and the results are shown in FIG. 10. The as-assembled system without annealing does not show a long-range ordered structure, as demonstrated by the broad rings in 2D pattern and the broad and few diffraction peaks of the structure factor. This Sys-Q7A₃₀ can be crystallized by annealing at 48° C. (about 10° C. below the M_{T}) for about 20 min. Upon further increasing temperature to 59° C., no structure was found, which means Q7 and Au nanoparticles can be re-dispersed in the solution after DNA de-hybridization. To investigate the thermal stability of STV on the QD surface, the system was further heated to 75° C. and kept at temperature for 1 hour. After cooling down to 26° C., the system again showed crystallization, which indicates the high thermal stability of capped STV. Such phase behavior is similar to the reported Au—Au systems and the above-described systems of Au—Au with different particle sizes.

The effects of biotin-DNA number (N) and particle ratio on the assembly phase behavior were also investigated. First the biotin-DNA number was changed while the mole ratio of QD to Au was maintained at 1:1. FIGS. 11A and 11B show the 2D SAXS pattern and corresponding S(q) for Sys-Q7A₃₀ with the mole ratio of biotin-DNA to Q7 (Au) as N=10 and 120, respectively. Compared with the structure factors of systems for N=10, 40 (FIG. 9C), and 120, one can conclude that a certain amount of biotin-DNA, at least ten times the Au particle amount, is required for good crystallization, but excess biotin-DNA seems not to frustrate the crystallization of the QD-Au system. Then the particle ratio was altered and the ratio of QD:Au:biotin-DNA was set at 1:2:80, 2:1:40, 10:1:20. After assembly, there were no visible particles in the supernatant for any of the above systems except the system with QD:Au:biotin-DNA as 10:1:20, which contains QD as easily observed by a UV lamp. The 2D SAXS patterns and S(q) corresponding to the above three systems are shown in FIGS. 11C and 11D. It was found that all the systems (pre-annealed) show similar structure factors and the particle ratios in this studied range don't have important effects on the structure. Therefore, as long as the QD and Au systems crystallize after annealing, they actually show the same structure, which may be in a global energy minimum state and thus independent of the initial state and assembly pathway.

To analyze the assembly structure, the peak position ratios were calculated. Since all the systems show the same structure, Sys-Q7A₅₀ was taken as the example. For this system, Qₓ/Q₁=1:1.36:2:2.63:3.19:3.9:4.8, and such values resemble the peak position ratios, which are 1:1.41:2:2.65:3.19:3.87:4.79, of diffraction planes (110), (200), (220), (321), (420), (521), (611) to (110) of a BCC structure. The relative scattering ability of Au to Q7 (Is_{Au}/Is_{Q7}) was calculated as ˜18. Supposing the assembly has a CsCl structure, the calculated results by the method used for Pd—Au system show that this system displays its intrinsic SC diffraction patter. The relative peak intensity of (100) to (110) is about 0.4, and so the first diffraction peak should be (100) and the peaks with Qₓ/Q₁ at 2.63, 3.9, and 4.8 should never appear, which contradicted the observed results. Therefore, either QD or Au should pack in a BCC structure, and the other one sit on some sites of this BCC frame.

Considering the relative intensity of (110) and (200) (I₍₁₁₀₎/I₍₂₀₀₎), a BCC with a sub-SC structure is proposed, where one kind of particle sits on BCC sites and combines with another type of particle to form an SC subunit. Such structure is a cubic La₂O₃-like structure (the high temperature X-phase described in Aldebert, P., et al., (Journal De Physique, 1979. 40(10): p. 1005-1012, which is incorporated herein by reference in its entirety), which corresponds to the Im ^{3̅}m space group with group number 229.

In this prototype structure, La sits in 2a sites, and O is randomly distributed over the 6b sites with a 50% probability that any one site is occupied. For an A-B particle system with this structure, and suppose particle A sits on 2a sites and 1 on 6b sites, the Is_{A}/Is_{B}-dependent diffraction behavior is calculated by PowderCell and the results are shown in FIG. 11F, where the two numbers correspond to A and B atom number and the square of their ratio is roughly equal to Is_{A}/Is_{B}. When Is_{A}>>Is_{B}, this structure shows a BCC diffraction pattern and the first peak is from (110) and has the strongest intensity. The I₍₁₁₀₎/I₍₂₀₀₎ decreases with the decrease of Is_{A}/Is_{B}; when Is_{A}=Is_{B}, this structure shows a SC diffraction pattern and I₍₁₁₀₎to becomes 0 while I₍₂₀₀₎ becomes strongest. With the further decrease of Is_{A}/Is_{B}, I₍₁₁₀₎ increases and I₍₁₁₀₎/I₍₂₀₀₎ becomes ˜0.4 when Is_{A}<<Is_{B}. According to the calculated results and Is_{Au}/Is_{Q7}, the Sys-Q7A corresponds to the case 80:20 in FIG. 11F, and Au and QD particles are on 2a and 6b sites, respectively. The I₍₁₁₀₎/I₍₂₀₀₎ of Sys-Q7A for short DNA spacers agrees with the calculated results, while this value decreases with spacer length and deviates from the calculated results. This spacer length-dependent intensity change may be related to the decreased correlation length with the increase of spacer length, which leads to diffraction (200) from subunit lattice stronger but (110) from unit lattice weaker. The proposed structure is also shown in FIG. 11F, where the QD was proposed to link two Au particles through its short axis direction since it can maximize the hybrid DNA number in this way.

Using the proposed La₂O₃ structure, D_{cc}M for Sys-Q7A are calculated using Q₁. For systems shown in FIGS. 9A through 9E, Q₁ are correspondingly 0.0230, 0.0223, 0.0201, 0.0152, and 0.0116 Å⁻¹, and using D_{cc}M=√2*π/Q₁, the D_{cc}M are correspondingly 19.3, 19.9, 22.1, 29.2, and 38.3 nm. For the calculation of D_{cc}C, the following parameters were used: R_{Au}=4.5 nm, R_{Q75}=7.5 nm (including STV), DNA number on Q7=20, and the other parameters are the same as that used for the Au—Au system. The calculated D_{cc}C for the above systems is accordingly as 20.5, 21.1, 23.2, 27.5, and 36.3 nm, which agrees with the D_{oc}M.

Two other kinds of QD with emission wavelengths centered at 605 nm (Q6) and 525 nm (Q5) were used to hybridize with Au nanoparticles. FIGS. 8C and 8D show the Iₚ(q), the fitting, and the size distribution for Q6 and Q5, respectively. The fitting results show that Q6 and Q5 also have elongated shape, and the long and short axis length are 11 nm and 5 nm for Q6, and 7 nm and 3 nm for Q5. Q6 and Q5 were accordingly hybridized with 9-nm Au nanoparticles to form Sys-QA6 and Sys-QA5, and the X_{A}-X_{B} sets were designated as 0-15, 15-15, 35-35, and 65-65, and the systems were denominated as Sys-QA6ₙ, and Sys-QA5ₙ, and n=15, 30, 50, and 80, respectively. FIGS. 12A through 12H give the 2D SAXS pattern and corresponding S(q) for Sys-QA6ₙ and Sys-QA5ₙ, and the images in FIGS. 12A through 12D and 12E through 12H correspond to n=15, 30, 50, and 80, respectively. All the systems were proposed to be La₂O₃ structure due to their similar S(q) to Sys-Q7A. With the decrease of QD size for QD-Au system, the I₍₁₁₀₎/I₍₂₀₀₎ increases, which agrees with the calculated S(q).

The Sys-QA6 and Sys-QA5 also have spacer length-dependent intensity change behavior. Q₁ was used to calculate the D_{cc}M. For Sys-QA6. Q₁ are 0.0238, 0.0209, 0.0182, and 0.01490 Å⁻¹, corresponding to n=15, 30, 50, and 80, respectively, and DₒₛM are correspondingly 18.6, 21.2, 24.3, and 29.6 nm. For Sys-QA5, Q₁ are 0.0245, 0.0205, 0.0187, and 0.0138 Å⁻¹, corresponding to n=15, 30, 50, and 80, respectively, and D_{cc}M are correspondingly 18.1, 21.6, 23.7, and 32.1 nm. For the calculation of D_{cc}C, the following parameters were used: R_{Au}=4.5 nm; R_{Q6S}=6.5 nm (including STV); R_{Q5S}=5.5 nm (including STV); DNA number on Q6 or Q5 is 20; and the other parameters are the same as that used for the Au—Au system. The calculated D_{cc}C for Sys-QA6ₙ are 19.8, 22.5, 26.6, and 35.6 nm for n=15, 30, 50 and 80, and for Sys-QA5ₙ, are 19.1, 21.9, 26.2, and 35.3 nm for n=15, 30, 50 and 80, respectively. D_{cc}C agrees with the D_{cc}M, especially for the short DNA spacer case.

The Au size effect on the Au-QD assembly structure was also investigated. Here, a system was constructed by assembling 16.6-nm STV-Au and Q7 and the X_{A}-X_{B} sets were designed as 15-15 and the system was denoted as Sys-Q7A16₃₀. FIGS. 13A and 13B show the 2D SAXS pattern and corresponding S(q) at 26° C. and 53° C., and FIG. 13C shows the 2D SAXS pattern, its Iₚ(q), and the fitting at 71° C. for the melting system. The Qₓ/Q₁ for this system at 53° C. is 1:1.73:2.38:3.2:3.92, and can be assigned to a BCC structure, which corresponds the case ˜80:1 in FIG. 11F. So this system also has a cubic La₂O₃-like structure.

Therefore, these results show that all the hybrid systems of QD and Au have cubic La₂O₃-like structures, and this is the first time that this kind of structure has been reported for bioassembled materials. Without wishing to be bound by the theory, it is considered that the elongated shape of QD is important for the formation of this novel structure, and we predict that other nanoparticles with similar shapes might result in the creation of similar assemblies.

The photoluminescence properties of the Au-QD systems were also measured. Take Sys-Q7A for the example. FIG. 14A shows the photoluminescence of Sys-Q7Aₙ, including the control system, with n=15, 30, 50, and 80 systems. It can be seen that the Au-QD systems show a distance-dependent fluorescence quenching behavior. The quenching efficiency (QE) of Sys-Q7A against the surface-to-surface distance between the QD and Au is given in FIG. 14B. The decay curve can be fitted by an exponential decay model where QE=QE₀·exp(−d/d₀) (see, e.g., Zheng, W. M. and L. He, Journal of Physical Chemistry C, 2010. 114(41): p. 17829-17835, which is incorporated herein by reference in its entirety), where QE₀ is the quenching efficiency when QD were directly attached to the Au surface, do is the distance constant within which fluorescence quenching occurs, and d the separating distance between the QD and Au surfaces. The fitting yielded an equation of QE=1.14·exp(−d/15.3), and the fitted d₀ agrees with the experimental values obtained by Zheng (2010).

### Example 12

### Au and Magnetic Iron Oxide Particles

About 9 nm thiolated DNA-capped Au nanoparticles and biotinylated DNA-capped IO nanoparticles were used as particle models to illustrate phase behavior for the hybrid system of Au and IO nanoparticles. The size and shape of the IO were characterized by TEM and SAXS. FIG. 15A shows the TEM image, FIG. 15B the SAXS Iₚ(q) and FIG. 15C the fitting of IO, which indicate that the IO have spherical shapes with diameters of 10.2±0.7 nm. For the Au and IO hybrid system, the ratio of IO to Au and biotin-DNA was set as 1:1:15, and the X_{A}-X_{B} sets were designed as 0-15, 15-15, 35-35, and 65-65, and the systems were nominated as Sys-IAₙ, with n=15, 30, 50, and 80, respectively.

FIGS. 16A through 16D give the 2D SAXS pattern and corresponding S(q) for Sys-IAₙ, and the images in FIGS. 16A through 16D correspond to n=15, 30, 50, and 80, respectively. Here, S(q) Iₐ(q)/Iₚ(q), Iₚ(q) was obtained from the melting system of Sys-IA₃₀. Two control systems (ICA-I and IAC-II) were designed, and IAC-I is the mixture of STV-IO and Au particles without biotin-DNA, and IAC-II is the mixture of STV-IO and biotin-DNA without Au particles. FIGS. 16E and 16F give the 2D SAXS pattern and corresponding S(q) for IAC-I and IAC-II, respectively. Both the control systems have aggregations and show similar S(q) and three peaks near about 0.033, 0.059, 0.102 Å⁻¹, which indicates that this structure actually comes from IO-IO aggregation. Such aggregation may be induced by the depletion attraction by biotin-DNA or DNA-Au particles because STV-IO doesn't form aggregates in solution as shown in FIG. 15A. Interestingly, these three peaks from IO disappear for short DNA spacers, such as n=15 and 30, but not for long DNA spacers like n=50 and 80. This result indicates that the DNA with shorter spacer has higher hybrid energy and thus can break the IO-IO aggregation to form IO-Au aggregation. Therefore, the Sys-IA consists of only IO-Au aggregation for short DNA spacers (n=15, 30), but IO-IO and IO-Au aggregation for long DNA spacers (n=50, 80).

The temperature-dependent phase behavior of Sys-IO was investigated. We found that Sys-IO showed different behavior, which depended on the spacer length. FIGS. 17A and 17B show the S(q) as a function of temperature for Sys-IA₃₀ and Sys-IA₅₀, respectively. For Sys-IA₃₀, when this system is heated to 55° C., all the diffraction peaks disappear, which means IO and Au nanoparticles in this system can be re-dispersed in the solution after DNA dc-hybridization. After cooling down the system, IO-Au hybrid structures form again, but it will take a long time (days) to form better structures. It should be noted that if the system is kept in the melting state (55° C.) for hours, the IO-IO aggregation will form and the aggregation has a structure similar to that shown in FIGS. 16E and 16F. But such IO-IO aggregation will eventually convert back into a IO-Au structure when the system cooled down. However, for Sys-IA₅₀, when this system is melted, only its first original peak disappears and the other peaks with Q at ˜0.033, 0.059, 0.102 Å⁻¹, which are the same peaks in IO-IO aggregation, still exist. This result again shows that Sys-IA₅₀, actually consists of two kinds of aggregations, namely, IO-IO and Au-IO, and also indicates that the kinds of IO-IO interaction are not related to the specific DNA hybridization.

The effects of biotin-DNA number (N) and particle ratio on the assembly phase behavior were investigated. First the biotin-DNA number was changed while the mole ratio of IO to Au was kept at 1:1. FIGS. 18A and 18B show the 2D SAXS pattern and corresponding S(q) for Sys-IA₃₀ with the mole ratio of biotin-DNA to IO (Au) as N=7 and 60, respectively. Compared with the structure factors of systems for N=15 (FIG. 16A) and the control system, one can conclude that an appropriate amount of biotin-DNA is required to break down the IO-IO aggregation and to form the IO and Au assembly. In the case of too little biotin-DNA it doesn't provide enough driving force for IO and Au assembly; while for too much biotin-DNA it may introduce excess depletion attraction that makes IO-IO aggregation uneasily broken as well. Then the particle ratio was changed and IO:Au biotin-DNA was set to 1:5:75 and 5:1:75. The 2D SAXS patterns and S(q) corresponding to the two systems are given in FIGS. 17C and 17D, and the results indicate that less IO than Au is not favorable for IO-Au assembly. This may be caused by the blocking of the IO surface DNA by excess Au particles due to the lower DNA coverage on IO (˜10 DNA on 10-nm IO).

A linker DNA was used to assemble STV-IO capped with biotin-DNA and Au particle capped with thiol-DNA. A linker system was designed as illustrated in FIG. 19, and the Lₙ spacer was designed as different number of poly T and the system was nominated as Sys-IALₙ. FIGS. 20A through 20D show the 2D SAXS pattern and corresponding S(q) for Sys-IALₙ, and the images in FIGS. 20A through 20D correspond to n=0, 30, 70, and 170, respectively. Compared with the IO-IO structure, the Sys-IAL shows peaks all coming from IO-Au aggregation for short DNA linkers (n=0), and shows peaks both coming from IO-IO and IO-Au for long DNA linkers (n=30, 70, 170). The linker system also shows similar temperature-dependent phase behavior to the direct hybrid system, namely, peaks from IO-Au rather than IO-IO aggregation disappear above melting temperature.

To analyze the assembly structure, the peak position ratios were calculated. Only systems displaying Au-IO peaks, such as Sys-IA₁₅, Sys-IA₅₀, and Sys-IAL₀, were used to calculate Qₓ/Q₁, and the ratio obtained was 1:1.7˜1.8:2.3˜2.5. A similar ratio was also obtained from other systems if the peaks from IO-IO were subtracted. According to the Qₓ/Q₁, the structure may be SC with Qₓ/Q₁ as 1:1.73:2.45 from diffraction planes (100), (111), (211), or BCC with Qₓ/Q₁ as 1:1.73:2.45 from diffraction planes (110), (211), (222), or FCC with Qₓ/Q₁ as 1:1.63:2.31:2.52 from diffraction planes (111), (220), (400), (331). For a binary SC system, the structure model can be CsCl, α-ReO₃, or AuCu₃; for a binary BCC system, the structure model can be La₂O₃; for a binary FCC system, the structure model can be NaTl, NaCl, ZnS (zincblende), or CaF₂. The Is_{Au}/Is_{IO} is calculated as 2.6 for Sys-Au-IO, and the proposed structure for CsCl, α-ReO₃, AuCu₃, La₂O₃, NaTl, NaCl, ZnS and CaF₂ with the corresponding calculated S(q) is accordingly shown in FIGS. 21A through 21H, respectively. The models, including CsCl with Q₁ from (110), AuCu₃ with Q₁ from (111), NaTl with Q₁ from (220), and ZnS with Q₁ from (11), seem possible in comparison of their relative peak intensity with experimental results. The D_{cc}M can be calculated as √6*π/Q₁, √6*π/Q₁, √6*π/Q₁, and 1.5*π/Q₁, for CsCl, AuCu₃, NaTl, and ZnS, respectively. For Sys-IAₙ, Q₁ are 0.0246, 0.023, 0.018, and 0.0135 Å⁻¹, corresponding to n=15, 30, 50, and 80, respectively, and D_{cc}M are correspondingly 31.3, 33.5, 42.8, and 55.8 nm for the CsCl, AuCu₃, and NaTl models, and 19.2, 20.5, 26 2, and 34.1 nm for the ZnS model. For Sys-IALₙ, Q₁ are 0.0213, 0.0177, 0.0156, and 0.015 Å⁻¹, corresponding to n=0, 30, 70, and 170, respectively, and D_{cc}M are correspondingly 36.1, 43.5, 49.3, and 51.3 nm for the CsCl, AuCu₃, and NaTl models, and 22.1, 26.6, 30.2, and 31.4 nm for the ZnS model.

To calculate the D_{cc}C for Sys-IAₙ, the following parameters were used: R_{Au}=4.5 nm; R_{IO}=9.5 nm (including STV); DNA number on IO-IO; and the other parameters are the same as that used for the Au—Au system. The calculated D_{cc}C for the Sys-IAₙ is 21.7, 23.7, 27.2, and 35.4 nm, corresponding to n=15, 30, 50, and 80, respectively. D_{cc}C agrees with the D_{cc}M of ZnS model. Therefore, our results show that all the hybrid systems of IO and Au have zincblende structures, and this is the first time that this kind of structure has been reported for bioassembled materials.

The magnetic field (B) effects on the phase behavior of the IO-Au hybrid systems were measured. Take Sys-IA₃₀ (a system having only IO and Au aggregation) and sys-IAL130 (a system containing a mixture of IO and Au aggregation and IO and IO aggregation system) for examples to illustrate such B effects. FIGS. 22A and 22B accordingly show the magnetic field-dependent 2D SAXS pattern and corresponding S(q) for Sys-IA₃₀ and Sys-IAL₁₃₀. For Sys-IA₃₀ with the increase of B, the third diffraction peak (331) of ZnS structure first disappears, and then the second peak (220) disappears, and finally only the first peak (111) survives at the highest B in this study. Interestingly, both the second and third peaks appear again after the removal of B, which indicates the B-dependent phase behavior is reversible. The sys-IAL₁₃₀ also shows a reversible B-dependent phase behavior. With the increase of B, the first peak (111) from IO-Au aggregation disappears, while the other peaks from IO-IO aggregation remain nearly constant. Such a change law of S(q) with B is similar to that of S(q) with T. For the above two systems, the first peak position nearly remains constant but its width becomes much broader with the increase of B, which indicates that the mean particle distance remains unchanged but the particles' position fluctuation increases. Such position fluctuation with B is related to the DNA stiffness. In comparison with Sys-IA₃₀, Sys-IAL₁₃₀ is easier to subject to this fluctuation due to its longer and more flexible DNA linker, and this leads to the loss of the first peak of Sys-IAL₁₃₀ while not of Sys-IA₅₀ at the same B.

### Example 13

### Au and Magnetic Iron Oxide Particles

In these systems, besides the DNA specific interactions between IO and Au, there are remarkable non-specific interactions, such as weak magnetic attraction and van der Waals interaction related to the limited DNA number, between IO nanoparticles. The assembly rules should be different from the Au—Au and Au—Pd systems. Moreover, a route with controllable interplay between the specific and non-specific interactions is promising for switchable structures. It was found that DNA-capped IO were ready to form aggregates. The S(q) is given in FIG. 34A (1), which corresponds to a system, denoted by Sys-FeO, containing IO nanoparticles capped with one type of 30-base biotinylated-DNA. The spectrum, denoted by Phase-F, shows two broad peaks centered at 0.033 and 0.059 Å⁻¹, respectively. This phase was assigned as a weak-ordered FCC structure, as indicated by the fit shown as black line in FIG. 34A (1). The Phase-F was triggered by the non-specific interactions, as evidenced by the temperature-dependent study, which displayed an absence of thermal dissociation process for such aggregates.

Interestingly, these non-specific interaction induced aggregates can switch into a binary component superlattice directed by DNA hybridization. FIG. 34A (2) shows a DNA length-dependent structure evolution of Phase-F by introducing direct complementary Au nanoparticles. In these direct hybridization systems, with N decrease from 145 to 85, 45, and 30, a new phase emerges accompanied by the consumption of the initial Phase-F. However, distinct from Phase-F, this new phase revealed a thermally reversible dissociation-association behavior, indicating that it was indeed a DNA-driven assembly by IO and Au nanoparticles. This new phase was denoted by Phase-D. The longer spacer systems for N=145 (FeO_Au₆₅_{_{—}}₆₅) and 70 (FeO_Au₃₅_{_{—}}₃₅) show a mixture phase-F and D, while shorter ones for N=45 (FeO_Au₁₅_{_{—}}₁₅) and 30 (FeO_Au₀_{_{—}}₁₅) show a pure phase-D as well as with improved structure order. This behavior is distinct from Pd—Au systems and Au—Au systems, where longer DNA favors better structure. This DNA-length dependent structure evolution seems universal for IO-Au systems and is also observed in the linker hybridization systems, which demonstrates that the longer linkers (N=90, 130, 190, and 230) produce a mixture phase of IO and IA, and shorter linker (N=60) gives the pure phase-D. Moreover, this binary IO and Au phase can transform back into IO phase by decreasing the DNA attraction forces. For example, upon keeping the Phase-D at Tₘ (typically −55° C.) for hours rather than cooling directly to room temperature, the Phase-F will form although this phase can eventually transformed into pure Phase-D if cooling down this system. Therefore, a switchable phase transition between different-component phases can be realized with the regulation of interplay between non-specific and specific interactions.

To further elaborate the DNA length effects on the phase switch behavior, the assembly kinetics of the two phases were analyzed upon introducing complementary Au nanoparticles into Phase-F. Two representative systems were investigated, including FeO_Au₁₅_{_{—}}₁₅ for assembly of pure Phase-FA and FeO_Au₃₅_{_{—}}₃₅ for assembly of mixed Phase-F and -D. Based on the time-dependent development of SAXS patterns, the ξ of the two phases was derived and plotted in FIG. 34C. In comparison with longer spacer system, the shorter one demonstrates a short time-scale for the development of Phase-D and a complete elapse of Phase-F at ˜40 h. The slower kinetics and smaller ξ for longer spacer system might be caused by the lower penetration capability into the Phase-F due to the higher entropic penalty, their lower effective DNA hybridization concentration, and the higher positional fluctuation due to more soft repulsive potentials.

Structural analysis suggested Phase-D with an Au nanoparticles-based FCC structure, where only Au nanoparticles show positional order. Compared with other types of possible lattices, such as CsCl, such FCC structure gives the best fit, as given by black solid line in FIG. 34A (2) for FeO_Au₀_{_{—}}₁₅. Due to the limited number of STV on the IO nanoparticles surface, the investigation of systems comprising Au and STV is helpful to understand the Phase-D structure. Two systems, namely STV_Au₁₅_{_{—}}₁₅ and STV_Au₀_{_{—}}₁₅ were constructed, and their S(q) are given in FIG. 34A (3). Interestingly, the STV_Au₁₅_{_{—}}₁₅ shows similar S(q) as phase-D, and meanwhile the similar fit quality was exhibited using the Au nanoparticles-based FCC structure. Different from FeO_Au₀_{_{—}}₁₅, the STV_Au₀_{_{—}}₁₅ could achieve a highly ordered state, which can be well fitted by such FCC structure. Above all, it's reasonable to such structure as a FCC for Au nanoparticles, which are surrounded by STV for STV-Au or IO for IO-Au as linker shells. The 3D and 2D schematics are illustrated FIGS. 34B and 34C, respectively.

Based on this FCC structure, the Dₛₛ for IO-Au DH systems was calculated and plotted as black spherical symbols in FIG. 34D. As the DC-model is applicable for the Au—Au and Pd—Au systems, it was also adopted to calculate Dₛₛ. Due to the linker roles for IO connecting two Au Nanoparticles, there could be variable angle (α), which formed between two adjacent Au-IO connections, as shown in FIG. 34D. Interestingly, it was found that Dₛₛ data fell in the calculations with α between 180° (upper straight line in FIG. 34D) and 109° (lower straight line in FIG. 34D). We then calculated α and gave the values in FIG. 3d, which showed that α changes from ˜170° to 109° with the increase of N from 45 to 145. This result implies that the IO shifts its position from a two Au Nanoparticles center to a three Au Nanoparticles (triangle) center or to a four Au Nanoparticles (tetrahedron) center with the increase of spacer number.

The magnetic response for IO-Au systems was also investigated. By changing the sample-magnet distance as shown in FIG. 34E, the magnetic field (B)-dependent response of two representative IO-Au systems were measured, namely, FeO_Au₁₅_{_{—}}₁₅ (Phase-D) and FeO_Au_{L130} (mixed Phase-F and -D). For FeO_Au₁₅_{_{—}}₁₅, the diffraction peaks became broader and even disappeared with B. The q₁ disappears for B at 0.11 T, and further increase of B to 0.16 T leads to the diminished q₂ and the residue of q₁. FeO_Au_{L130} shows a more profound B-response for Phase-FA and an inert response for Phase-F. That is q₁ from Phase-D disappears for B at 0.16T and other peaks from Phase-F display subtle changes. The S(q) can convert back to the initial states for the both systems, indicating a reversible B response. The softer potential from longer DNA and lower hybridization efficiency might be responsible for the more responsive behavior of FeO_Au_{L130} system. This result suggested that through rational DNA design, one can fabricate systems with B-response switchable superlattice of different states, which could be interesting for smart responsive materials.

### Example 14

### Au and Protein (Sireptavidin)

Nine-nm thiolated DNA-capped Au nanoparticles were used to hybridize with streptavidin (STV). The ratio of STV to Au and biotin-DNA was set as 1:20:100, and the X_{A}-X_{B} sets were designed as 0-15, 3-15, 15-15, and 35-35, and the systems were nominated as Sys-SAₙ, with n=15, 18, 30, and 50, respectively.

FIGS. 23A through 23D give the 2D SAXS pattern and corresponding S(q) for Sys-SAₙ, and the images in FIGS. 23A through 23D correspond to n=15, 18, 30, and 50, respectively. Here, S(q)=Iₐ(q)/Iₚ(q), Iₚ(q) was obtained from the melting system. The Qₓ/Q₁ were calculated as 1:(1.69˜1.78):(2.3˜2.5):(2.7˜2.8):(3.1˜3.2). According to the Qₓ/Q₁, the structure can be SC with Qₓ/Q₁ as 1:1.73:2.45:2.83:3.16 from diffraction planes (100), (111), (211), (220), and (310) or BCC with Qₓ/Q₁ as 1:1.73:2 A5:2.83:3.16 from diffraction planes (110), (211), (222), (400), and (420) or FCC with Qₓ/Q₁ as 1:1.63:2.31:2.52:2.83:3.26 from diffraction planes (111), (220), (400), (331), (422), and (440). For a binary SC system, the structure model can be CsCl, α-ReO₃, or AuCu₃; for a binary BCC system, the structure model can be La₂O₃; for a binary FCC system, the structure model can be NaTl, NaCl, ZnS (zincblende), or CaF₂. The Is_{Au}/Is_{STV} is roughly considered as ∞, and according to the calculated results the possible models are La₂O₃, NaCl, ZnS, and CaF₂. The D_{∞}M can be calculated as √2*π/Q₁, √3*π/Q₁. 1.5*π/Q₁, and 1.5*π/Q₁ for La₂O₃, NaCl, ZnS, and CaF₂, respectively. For Sys-SAₙ, Q₁ are 0.0288, 0.0273, 0.0246, and 0.0225 Å⁻¹, corresponding to n=15, 18, 30, and 50, respectively, and D_{cc}M are correspondingly 15.4, 16.3, 18.1, and 21.2 nm for the La₂O₃ models, 18.9, 19.9, 22.1, and 25.9 nm for the NaCl model, and 16.4, 17.3, 19.2, and 22.4 nm for the ZnS and CaF₂ models.

To calculate the D_{cc}C for Sys-SAₙ, the following parameters were used: R_{Au}=4.5 nm, R_{STV}=3 nm (including STV), DNA number on STV=4, and the other parameters are the same as that used for the Au—Au system. The calculated D_{cc}C for Sys-IAₙ is 15.9, 16.4, 18.1, and 21.6 nm, corresponding to n=15, 18, 30, and 50, respectively. D_{cc}C agrees with the D_{cc}M of the La₂O₃, ZnS and CaF₂ models. Since STV doesn't give sufficient scattering, the precise location of the organic compound of the assembly can't be predicted, and the predicted La₂O₃, ZnS and CaF₂ structures are based on the positions of the Au particles. However, considering STV has four binding sites for biotin, the most likely model is the CaF₂-like crystalline organization.

### Example 15

### QD and QD

STV-Q7 was used to hybridize with STV-Q7 and STV-Q5 to form Sys-Q77 and Sys-Q75 systems, respectively. The ratio of QD to QD and biotin-DNA was set as 1:1:40, and the X_{A}-X_{B} sets were designed as 3-3, 0-15, and 15-1 for Sys-Q77, and the systems were denoted Sys-Q77ₙ, with n=3, 15, and 30, respectively. The X_{A}-X_{B} sets were designed as 3-3, 15-15, and 35-35 for Sys-Q75, and the systems were denoted Sys-Q75ₙ, with n=3, 30, and 50, respectively.

FIGS. 24A through 24F give the 2D SAXS pattern and corresponding S(q) for Sys-Q77ₙ and Sys-Q75ₙ, and the images in FIGS. 24A through 24C correspond to n=3, 15, and 30 for Sys-Q77, and the images in FIGS. 24D through 24F correspond to n=3, 30, and 50 for Sys-Q75ₙ, respectively. Here, S(q)=Iₐ(q)/Iₚ(q), Iₚ(q) was obtained from the corresponding melting system. The Qₓ/Q₁ were calculated as 1:(1.76˜1.85):(2.65˜2.73):(˜3.4). According to the Qₓ/Q₁, and the above analysis for Au—Au and Pd—Au systems, the structure can be either the CsCl or the NaTl structure. The D_{cc}M for these two structure can be calculated as √6*λ/Q₁. For Sys-Q77ₙ, Q₁ are 0.0288, 0.0282, and 0.0258 Å⁻¹, corresponding to n=3, 15, and 30, respectively, and D_{cc}M are correspondingly 26.7, 27.2, and 29.8 nm. For Sys-Q75ₙ, Q₁ are 0.0312, 0.0258, and 0.0234 Å⁻¹, corresponding to n=3, 30, and 50, respectively, and D_{cc}M are correspondingly 24.7, 29.8, and 32.9 nm.

To calculate the D_{cc}C for Sys-QDₙ, the following parameters were used: R_{Q2}=12 nm (long axis size including STV), R_{Q5}=10 nm (long axis size including STV), DNA number on QD=20, and the other parameters are the same as that used for the Au—Au system. The calculated D_{cc}C for the Sys-Q75ₙ is 25.1, 29.1, and 32.1 for n=3, 15, and 30, respectively, and for the Sys-Q77ₙ is 26.8, 28.2, and 30.6 for n=3, 30, and 50, respectively. D_{cc}C agrees with the D_{cc}M of both the CsCl and NaTl models, so the possible structures for STV and Au system are CsCl and NaTl.

The photoluminescence (PL) properties of the QD-QD systems, including Sys-Q77 and Sys-Q75, were measured. FIG. 25A shows the photoluminescence of Sys-Q77ₙ, including the control system (a mixture of Q7 and Q7 without biotin-DNA), and n=18, 30, and 50 systems. Different from the Au-QD systems, the Q7-Q7 systems show a distance-dependent fluorescence-enhancing behavior. The enhancement factor (EF) of Sys-Q77 against the surface-to-surface distance between Q7 and Q7 is given in FIG. 25B. EF=(Iₙ−I_{c})/I_{c}, where Iₙ and I_{c} correspond to the PL intensity of Sys-Q77ₙ and the control system, respectively. The EF is inversely proportional to the surface-to-surface distance. FIG. 25C shows the photoluminescence of Sys-Q75ₙ, including the control system (a mixture of Q7 and Q5 without biotin-DNA), and n=18, 30, and 50 systems. Such Q7-Q5 systems show a distance-dependent fluorescence quenching of Q5 and enhancing of Q7 behavior. The enhancement-to-quenching factor (EQF) of Sys-Q75 against the surface-to-surface distance between Q7 and Q5 is given in FIG. 25D. EQF=(Rₙ−R_{c})/R_{c}, where R=I₇₀₅/I₅₂₅, and the subscript n and c denotes respectively Sys-Q75ₙ, and the control system, and I₇₀₅ and I₅₂₅ correspond to the PL intensity at 705 nm and 525 nm of Sys-Q75, respectively. It can be seen that the EQF is proportional to the surface-to-surface distance.

### Example 16

### QD and Pd

STV-Q7 was used to hybridize with STV-Pd NDs to form the Sys-QPD system. The ratio of QD to Pd and biotin-DNA was set as 1:1:40, and the X_{A}-X_{B} sets were designed 3-3, 15-15, and 35-35, and the systems were nominated as Sys-QPDₙ, with n=3, 30, and 50. FIG. 26 gives the 2D SAXS pattern and corresponding S(q) for Sys-QPD₃₀ at different temperatures. This system show a similar temperature-dependent phase behavior as system IO-Au with long DNA spacers, and the Pd particles can't be re-dispersed into solution. This system doesn't have long-range order since it actually only shows one peak.

The photoluminescence properties of the Pd-QD systems were also investigated. FIG. 27 shows the photoluminescence of Sys-QPDₙ, including the control system (a mixture of STV-PD and Q7 without biotin-DNA) and n=3 and 50 systems. Similar to the Au-QD systems, the Pd-QD systems also show a distance-dependent fluorescence quenching behavior. To compare the QE of these two systems, we selected Sys-QPD₅₀ and Sys-Q7A₅₀, and found that the QE of Sys-QPD₅₀ and Sys-Q7A₅₀ was 0.26 and 0.14, respectively. Additionally, the particle surface-to-surface distance in Sys-QPD₅₀ is bigger than that in Sys-Q7A₅₀ due to an additional STV on PD surface. Therefore, in comparison to the Au-QD systems, the Pd-QD systems show a more profound distance-dependent fluorescence quenching behavior.

STV-IO was used to hybridize with STV-IO to form the Sys-II system. The ratio of IO to IO and biotin-DNA was set as 1:1:15, and the X_{A}-X_{B} sets were designed as 15-15, and the systems were nominated as Sys-II₃₀. FIG. 24 gives the 2D SAXS pattern and corresponding S(q) for Sys-II₃₀. This system shows very broad peaks and doesn't have long-range order.

### Example 18

### QD and Au

These systems comprised QD and Au nanoparticles. Specifically, the 3D assembly of Au nanoparticles with three types of QD, namely QD705 (Q7), QD605 (Q6), and QD525 (Q5). Due to the comparable DNA grafting number (f) and hydrodynamic radius between QD and Au, a CsCl superlattice similar to Au—Au system would be expected. However, additional detailed structure information, such as compositional disorder, can be studied in the QD-Au systems thanks to the remarkable ΔP(q) but similar effective size for Au and component/size-tunable QD.

Similar to Pd nanoparticles, the DNA-grafted QDs are well-dispersed and stable in an aqueous solution. The S(q of three DH systems with N=30, namely, Q7_Au₀_{_{—}}₁₅, Q6_Au₀_{_{—}}₁₅, and Q5_Au₀_{_{—}}₁₅, are given in FIG. 35A. Their S(q) displayed similar peak ratios and were assigned as SC patterns for the binary CsCl structures. Similar to the Au size-dependent S(q) evolution behavior in Pd—Au systems, as the QD changes from ˜2 nm CdSe/ZnS to ˜6 nm CdTe/ZnS, the intensity ratio of (110) to (100) increases caused by the decrease of ΔP(q) between QD and Au NPs, which features the binary CsCl lattice. A fit for Q7_Au₀_{_{—}}₁₅ using CsCl lattice is given as black line in FIG. 35A.

The interparticle distances in these QD and Au binary systems can also be facilely tuned by regulating DNA length. The N was varied from 30 to 33, 45, 85, and 145 for all these three types of binary superlattices. FIG. 35B (top) gives the S(q) of three representative Au-Q7 systems, including Q7_Au₀_{_{—}}₁₅, Q7_Au₃₅_{_{—}}₃₅, and Q7_Au₆₅_{_{—}}₆₅. The Dₛₛ of these QD-Au systems was calculated from the SAXS data and summarized the results as symbols in FIG. 35D, which exhibited that the Dₛₛ can be tuned from ˜12 to 31 nm. For short DNA length systems, supertattices comprising larger size QD display smaller Dₛₛ, which consists with the DC-model predicted tendencies (line in FIG. 35D). However, similar Dₛₛ were observed for all types of QD-Au superlattices with long DNA, and this might be related to compositional disorder.

Similar to Au—Pd systems, the structural order was improved with N and the ξ increased from 82 nm to 168 nm with the increase N from 30 to 145. Besides the increase of ξ, the first two peak intensity ratio (I₂/I₁) also increases with N regardless of types of QD. Such intensity modulation does not result from different nanoparticles size in systems for Pd hybridized with Au of different sizes and Au with QD of different types. This S(q) evolution results from the compositional order-to-disorder (OTD) transition in the binary CsCl structures. Such OTD transition has been extensively studied in atomic systems, such as ZnCu alloys, and recently was demonstrated in a computational work on DNA-assembled Au systems, which show a ODT transition with elevated temperature approach Tₘ. The ODT process can be described by means of a long-range order parameter η, defined as η=(r_{A}−F_{A})/(1−F_{A}), where r_{A} is fraction of A sites occupied by the “right” particles, i.e. A particles, and F_{A} is fraction of A particles in the lattice. The value of r_{A}=1, η=1 and r_{A}=F_{A}, η0 respectively correspond to compositional ordered and disordered lattice.

In the case of a CsCl lattice, such a transition is schematically illustrated in FIG. 35C, which implies that a diffraction pattern evolution from SC to BCC will emerge as η decreases from 1 to 0. With increasing DNA length in our QD-Au systems, the gradual increase of I₂/I₁, values, a feature for SC to BCC pattern evolution, indicates a smaller η for longer DNA systems. The increase of softness of interparticle repulsive potential with DNA length might be responsible for this ODT transition. It's also reasonable that the Dₛₛ becomes similar for longer DNA Au-QD systems regardless of QD kinds because Au and QD are more like one type of “average” nanoparticle in the superlattice for smaller η. Obviously, such OTD transition can be only observed in heterogeneous systems, comprising components with quite different P(q), which could explain the absence of such ODT transition in our Pd (˜11 nm)-Au (˜10 nm) systems. The dark lines in FIG. 35B give the fit using CsCl lattice with and without consideration of compositional order. By the fit, the N-dependent η for Q7-Au systems was obtained and plotted in FIG. 35C, which shows that η decreases from ˜0.98 to ˜0.54 with N increase from 30 to 145.

The N-dependent compositional and structural order behaviors hints to a certain balance between DNA flexibility and rigidity that is crucial for ordering. According to this guidance, a modified-linker hybridization system was designed, denoted by Q7_Au_{1.30C24}, where the central 24-base segments of the 30-base linker part in Q7_Au_{L30} are hybridized into a rigid duplex. FIG. 35C (Bottom) gives the S(q) and the CsCl lattice fit for this system. Indeed, Q7_Au_{1.30C24} shows large ξ(>700 nm) and highly improved crystalline quality.

The photoluminescence (PL) properties of the QD-Au systems were also examined. FIG. 4E gives a set of steady-state and time-resolved PL spectra collected from Q7_Au DH systems, including N change from 145 to 85, 45 33 and 30, and a free dispersed biotinylated DNA-capped Q7 solution. A progressive PL quenching of QD is clearly observed as decreasing N, especially for N in the range from 45 to 30. In comparison with free QD, the PL intensity of superlattice decreases by about 8%, 20%, and 60% for N=145, 45, and 30, respectively. The lifetime (i) also progressive decreases from 62.1 as for free QD to 59.2 ns, 44.5 ns and 16.6 ns for superlattice accordingly corresponding to N=130, 30, and 15. The quenching efficiency, E=1−(τₛ/τ_{f}), where τₛ and τ_{f} are accordingly the lifetime of QD in the superlattice and free-states, reached ˜0.74 for the system with N=30, which the Dₛₛ is ˜12 nm.

### Example 19

### Binary Systems Without Au

The arbitrary binary combination of different types of QD (Q7, Q6, and Q5), different shape of Pd (PD, PC, PO), and IO were investigated. It was found that the grafting DNA number on the nanoparticles (f) plays a crucial role for assembly behavior. For example, QD of each three types (with f 20˜40) and Pd of each three shapes (with f˜15˜25) can hybridized with other into a superlattice, but the systems containing IO nanoparticles (with f˜3˜8) only form non-specific induced clusters with the size typically less than 100 nm. All these systems display thermally reversible dissociation-association behaviors, implying the DNA-directed assembly. Structural analysis indicates that all these superlattices can be assigned with CsCl lattices but of quite different degree of structure order.

The PL behaviors of QD-based binary systems was also investigated. The lifetime is summarized in FIG. 35F. The superlattice shows an energy transfer process, where involves ˜20% decrease in donor lifetime and ˜12% increase in acceptor lifetime in comparison with free particles. The current studies on fluorescence behavior of QD near metal NPs and QD most focused on clusters, the present QD-Au and QD-QD superlattice provide a platform to study the collective optical properties in 3D lattice due to their well-controlled structural ordering and lattice parameter.

### Example 20

### Summary of DNA-Mediated Assembly

The phase diagram for the assembled systems is summarized in FIG. 36A. Based on all the systems investigated, several important factors of the phase behavior of heterogeneous binary ˜10 nm NP-A and B systems are derived. i) Two threshold values, f_{T1} and f_{T2}, are accordingly required for the assembly of particle into micro-scale (or to form participate in solution) and into well ordered crystals. As plotted in FIG. 36A, if the two components have similar grafting DNA number, f_{T1} is about 20 for 10 nm NPs; and n_{T} could be relaxed to ˜3-8 if the other component possesses a high DNA number. i.e. f_{T1}˜60 for 10 nm NPs in the experimental limit, f_{T2} is about 30, ii) For systems involving NP-A (for example IO) with considerable non-specific interactions, besides the required f_{T1} for NP—B short length DNA is necessary to break and transform the non-specific interactions-induced aggregates into DNA-driven dominant assemblies. iii) For anisotropic shaped NPs involved systems, more spherical-like NPs (e.g Pd-dodecahedrons) generate better structure order of superlattices. v) For systems involving one type NPs with high f (e.g. Au) and the other with low f (e.g. QD), shorter (rigid) DNA benefits the compositional order and longer (flexible) DNA favors better structure order. A deliberate balance between DNA rigidity and flexibility is crucial for improving the ordering degree. This behavior is different for systems with high f for both types NPs, and in those systems DNA flexibility is the most important factors for ordering. If both components possess low f, a compositional disorder is favorable even for short DNA, as indicated in QD and Pd systems that the first peak diffracted from (110) planes.

The quantitative structural analysis demonstrated the interparticle center-to-center distances (D_{cc}) of any binary systems can be predicted from their corresponding single systems. Taking DH systems with N=30 for example, the effective Rₑ (=D_{cc}/2) of five components were calculated, including Au, Q7, Q5, PD, and PC NPs based on the SXSA data from single component systems. The Rₑ of each component is represented by black and gray bars in FIG. 5b. These data agree well with the DC models. The values simply by the sum of two Rₑ agree well with the D_{cc} (represented by solid narrow bars in FIG. 36B) obtained from the corresponding binary systems. Such consistency might help one to distinguish the lattice types because of the dependence of D_{cc} on lattice types with known SAXS data, and also benefit the understanding of DNA configurations on NPs surfaces as well as between nanoparticles.

In summary for the assembly part described in these examples, several examples for DNA-mediated assembly of binary systems have been presented, which show rich phases, such as Au and Au with both same and different size forming a NaTl structure, Au and Pd most likely forming a NaTl structure, Au and QD forming a La₂O structure, Au and IO forming a zincblende structure, Au and STV most likely forming a CaF₂ structure, QD and QD with same/different size forming a NaTl or CsCl structure, and the system comprised of Pd and QD or IO and IO that most likely having an amorphous phase. Although the examples shown are for binary systems, the use of DNA to assemble three or more multi-component systems is also possible.

Data on the fluorescence properties of metal (Au, Pd) and fluorescent particle (QD) systems, QD and QD systems, and the magnetic field effects on the phase behavior of a metal (Au) and magnetic particle (IO; Fe₂O₃) system were also obtained. All the measured metal-fluorescent systems showed a distance-dependent fluorescence quenching behavior. In comparison to the Au-QD systems, the Pd-QD systems showed a more profound quenching effect. For QD and QD systems, the system with same types of QD showed a distance-dependent fluorescence enhancement behavior, and the system comprised of different types of QD showed a fluorescence quenching for small QD and enhancement for big QD. Metal-magnetic particle systems show a reversible magnetic field intensity, modulation phase behavior.

Other and further embodiments utilizing one or more aspects of the inventions described above can be devised without departing from the spirit of Applicant's invention. For example, specific embodiments have been described using gold and palladium nanoparticles of an approximate diameter of about 10 nm, but particles of other materials (metallic, semi-conductive, magnetic, dielectric, etc.) of various dimensions may be substituted and still be within the confines of this disclosure. In addition, although the examples have, for purposes of concreteness, been described with reference to DNA functionalization, micro- and nano-objects can be functionalized similarly in accordance with the methods of the present disclosure using RNA or PNA, as both RNA and PNA have the same addressable properties as does DNA, and similar melting temperatures and structure. PNA is artificial and is therefore more resistant to degraedation than is DNA, allowing it to be used under conditions inimical to DNA, including but not limited to non-aqueous solvents. Further, DNA and RNA may be used in concert, as appropriate. Further, the various methods and embodiments of the functionalization of DNA as described herein can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to fully protect all such modifications and improvements that come within the scope or range of equivalent of the following claims.

1. A DNA-nanoparticle conjugate, comprising:
a functionalized hydrophilic, hydrophobic, or neutral nanoparticle;
a protein covalently bound to the functionalized nanoparticle; and
DNA biotinylated to the protein.

2. (canceled)

3. The DNA-nanoparticle conjugate of claim 1, wherein the protein is streptavidin.

4. The DNA-nanoparticle conjugate of claim 1, wherein the covalent bond between streptavidin and the functionalized nanoparticle is an amide bond.

5. The DNA-nanoparticle conjugate of claim 1, wherein the nanoparticle is functionalized with a mercapto acid ligand.

6. The DNA-nanoparticle conjugate of claim 5, wherein the mercapto acid ligand is a mercaptoundecanoic acid.

7. The DNA-nanoparticle conjugate of claim 1, wherein the nanoparticle is functionalized with an amphiphilic polymer.

8. The DNA-nanoparticle conjugate of claim 7, wherein the amphiphilic polymer is a lipid-PEG carboxylic acid.

9. The DNA-nanoparticle conjugate of claim 1, wherein the functionalized nanoparticle comprises a magnetic material, a plasmonic material, a photonic material, a catalytic material, and a biological material.

10. (canceled)

11. The DNA-nanoparticle conjugate of claim 9, wherein the magnetic material is Fe₂O₃, the photonic material is a quantum dot selected from CdSe/ZnS or CdTe/ZnS, the catalytic material is selected from Pd or Pt, the plasmonic material is selected from Au, and the biological material is a protein.

12. (canceled)

13. (canceled)

14. (canceled)

15. (canceled)

16. The DNA-nanoparticle conjugate of claim 11, wherein the functionalized nanoparticle has a shape of octahedron, cube or dodecahedron.

17. The DNA-nanoparticle conjugate of claim 1, wherein a number of DNA molecules attached to the nanoparticle is at least 3.

18. The DNA-nanoparticle conjugate of claim 17, wherein the number of DNA molecules attached to the nanoparticle ranges between 3 and 60.

19. The DNA-nanoparticle conjugate of claim 17, wherein a length of DNA attached to the nanoparticle ranges between 30 and 180 nucleotide bases.

20. A three-dimensional (3D) ordered superlattice comprising a plurality of DNA-nanoparticle conjugates assembled into one or more superlattices by a direct or linker-mediated hybridization, wherein the DNA-nanoparticle conjugate, comprises: a functionalized hydrophilic, hydrophobic, or neutral nanoparticle; a protein covalently bound to the functionalized nanoparticle; and DNA biotinylated to the protein.

21. The three-dimensional (3D) ordered superlattice of claim 20, wherein the number of the hybridized bases between complementary DNA is 15.

22. (canceled)

23. The three-dimensional (3D) ordered superlattice of claim 20, wherein the protein is streptavidin.

24. The three-dimensional (3D) ordered superlattice of claim 20, wherein the covalent bond between streptavidin and the functionalized nanoparticle is an amide bond.

25. The three-dimensional (3D) ordered superlattice of claim 20, wherein the nanoparticle is functionalized with a mercapto acid ligand.

26. The three-dimensional (3D) ordered superlattice of claim 20, wherein the mercapto acid ligand is a mercaptoundecanoic acid.

27. The three-dimensional (3D) ordered superlattice of claim 20, wherein the nanoparticle is functionalized with an amphiphilic polymer.

28. The three-dimensional (3D) ordered superlattice of claim 20, wherein the amphiphilic polymer is a lipid-PEG carboxylic acid.

29. The three-dimensional (3D) ordered superlattice of claim 20, wherein the nanoparticle comprises a magnetic material, a plasmonic material, a photonic material, a catalytic material, or a biological material, and has a shape of an octahedron, a cube or a dodecahedron.

30. The three-dimensional (3D) ordered superlattice of claim 20, wherein the magnetic material is Fe₂O₃, the photonic material is a quantum dot selected from CdSe/ZnS or CdTe/ZnS, the catalytic material is selected from Pd or Pt, the plasmonic material is selected from Au, and the biological material is a protein.

31. (canceled)

32. The three-dimensional (3D) ordered superlattice of claim 20, wherein at least one nanoparticle within the superlattice is made from a material different than at least one other nanoparticle within the same superlattice.

33. The three-dimensional (3D) ordered superlattice of claim 29, wherein at least one nanoparticle within the superlattice is palladium (Pd) and at least one other nanoparticle within the superlattice is gold (Au), wherein at least one nanoparticle within the superlattice is iron oxide (Fe₂O₃) and at least one other nanoparticle within the superlattice is gold (Au), or wherein at least one nanoparticle within the superlattice is a CdSe/ZnS or CdTe/ZnS quantum dot and at least one other nanoparticle within the superlattice is gold (Au).

34. (canceled)

35. (canceled)

36. The three-dimensional (3D) ordered superlattice of claim 20, wherein a number of DNA molecules attached to the nanoparticle ranges between 3 and 60 and a length of DNA attached to the nanoparticle ranges between 30 and 180 nucleotide bases.

37. (canceled)

38. A method of functionalizing hydrophilic or hydrophobic nanoparticles with DNA, the method comprising:
synthesizing hydrophilic or hydrophobic nanoparticles under conditions suitable to generate said nanoparticles having a substantially uniform size and shape;
contacting said nanoparticles with reagents under conditions suitable to replace or add carboxylic acid functional groups to the ligands in a ligand-exchange process;
contacting the nanoparticle surface with a protein under covalent bond-forming reaction conditions for a period of time sufficient to conjugate the protein onto the nanoparticle surface; and
contacting the protein on the nanoparticle surface with biotinylated-DNA.

39. (canceled)

40. (canceled)

41. The method of claim 38, wherein the protein is streptavidin.

42. A method of DNA functionalization of a nanoparticle, the method comprising:
grafting to the nanoparticle a ligand having an exposed carboxylic group;
conjugating streptavidin having a plurality of exposed amine groups to the ligand grafted to the nanoparticle to form a covalent amide bond; and
attaching a biotinylated DNA to the streptavidin.
